(19) United States
(12) Patent Application Publication
(10) Pub. No.: US 2004/0197335 A1
(43) Pub. Date: Oct. 7, 2004

(54) Non-myeloablative tolerogenic treatment with tyrphostins

(76) Inventors: Shimon Slavin, Jerusalem (IL); Alexander Levitzki, Jerusalem (IL); Aviv Gazit, Jerusalem (IL); Shoshana Morecki, Jerusalem (IL)

Correspondence Address: Anthony Castorina, G E Ehrlich, Suite 207, 2001 Jefferson Davis Highway, Arlington, VA, 22202, US

(21) Appl. No.: 10/479,523

(22) PCT Filed: Jun. 16, 2002
(86) PCT No.: PCT/IL02/00467

(51) Int. Cl. A61K 39/395; A61K 39/00

(52) U.S. Cl. 424/155.1; 424/184.1; 424/277.1

(57) ABSTRACT

A method of inducing immune tolerance in a first mammal to antigens of a second, non-syngeneic, mammal, is disclosed. The method is utilized to minimize graft rejection and/or reduce graft-versus-host diseases in transplantation procedures and to produce hematopoietic mixed chimeras. Methods of determining the activity of tyrphostins and the optimal concentration thereof in this method are also disclosed.

### FIELD AND BACKGROUND OF THE INVENTION

The present invention relates to a method of inducing donor-specific tolerance in a recipient and, more particularly, to an administration of a tolerogenic treatment to a recipient mammal prior to transplantation of a donor-derived transplant therein. The tolerogenic treatment of the present invention comprises administration of non-syngeneic donor antigens into a recipient mammal and eliminating those recipient T lymphocytes responding to the donor antigens, using a non-myeloablative dose of a tyrphostin. The present invention can hence be used to prevent transplant rejection and/or prevent the development of a graft versus host disease (GVHD), by the generation of unilateral or bilateral immune tolerance prior to transplantation.

The terms “host” and “recipient” are used herein interchangeably.

The terms “graft” and “transplant” are used herein interchangeably.

Transplantation of organs, hematopoietic cells and somatic cells has been a crucial therapeutic regimen for patients suffering from a variety of maladies. Although the techniques necessary for transplants are quite straightforward, the great stumbling block for successful transplantation has been the immune system. A fundamental problem has been the great vigor with which the recipient immune system reacts against introduction of antigens found in donor tissues or cells. Another problem, limited to transplantation of hematopoietic cells and hematopoietic cells rich organs (e.g., small intestine), is the development of a graft versus host disease (GVHD).

Transplantation of allogeneic donor (i.e., the same species but not genetically identical to the recipient) or xenogeneic donor (i.e., a species other than that of the recipient) grafts has posed particularly great difficulties. The continued functioning of any donor graft depends upon continued functioning of the donor cells that make up that graft. The cells of donor grafts, however, can elicit an immune reaction on the part of the recipient which, if unchecked, may lead to destruction of the graft.

One method of alleviating the reaction by the recipient against a graft has been administration of immunosuppressive treatment to the recipient. Unfortunately, despite the availability of new and very effective immunosuppressive drugs, recurrent episodes of acute and chronic graft rejection remain common, frequently causing loss of graft function. Moreover, the long-term success of transplantation is often limited by complications resulting from drug-related toxicity and from long-term immunosuppression (e.g., infections and secondary malignancies). In addition, transplantation of bone marrow cells (BMC) or small intestine, which are rich in immunocompetent lymphocytes, is frequently associated with a potential life-threatening complication due to graft versus host disease (GVHD).

It has been shown that a full hematopoietic chimera, i.e., a patient whose own BMC have been 100% replaced by permanently engrafted BMC from another individual (donor), can permanently accept donor-derived allografts with no need for maintenance immunosuppressive therapy. However, induction of full hematopoietic chimerism has been difficult to accomplish. First, substantially complete destruction of the recipient's immunohematopoietic compartment (“lethal” conditioning) is usually required for engraftment of matched and especially mismatched BMC. With lethal conditioning of the recipient, GVHD consistently causes morbidity or mortality. In such cases, T-cell depletion of the graft hematopoietic material represents the only approach for effective prevention of GVHD. T cell depletion is also effective in preventing graft versus malignancy (GVM) effects, or other graft related, non-malignant, diseases such as genetic diseases, diseases caused by deficiency of stem cell products or autoimmune diseases. T-cell depletion in turn is associated with an increased incidence of graft rejection. To overcome the problem of graft rejection, recipients of T cell depleted marrow allografts may require particularly strong conditioning or, alternatively, very high numbers of T cell depleted BMC. Subjecting patients to aggressive rejection-prevention protocols, such as total body irradiation (TBI) alone or TBI in combination with a short course of immunosuppressive drugs is unlikely to be accepted by clinicians treating patients in need of organ allografts.

It has been proposed that true bilateral tolerance associated with mixed donor/recipient hematopoietic chimerism, i.e., the condition in which a patient possesses both recipient and donor hematopoietic stem cells, rather than with full chimerism, would be preferable in clinical organ transplantation. Several experimental protocols have been designed to induce transplantation tolerance leading to mixed chimerism. Conditioning has required the use of high dose TBI followed by infusion with a mixture of T cell depleted donor and recipient BMC (Sachs et al., Ann. Thorac. Surg., 56:1221 (1993); Ildstad et al., Nature, 307:168 (1984)), or inoculation with donor BMC after lower dose TBI and infusion of a mixture of antibodies against CD4⁺ T cells, CD8⁺ T cells and NK cells leading to general pancytopenia (Tomita et al., J. Immunol., 153:1087 (1994); Tomita et al., Transplantation, 61:469 (1996)).

An alternative approach that involves irradiation with a sublethal dose of TBI and inoculation with a very high number of T cell depleted donor-derived hematopoietic cells has been developed (Reisner et al., Immunol. Today, 16:437 (1995); Bachar-Lustig et al., Nature Medicine, 12:1268 (1986)). Tolerogenic treatments using cyclophosphamide (hereinafter also referred to as “Cytoxan” or “Cy”) in combination with TBI have also been described.

Total lymphoid irradiation (TLI) has been employed successfully as the sole preparatory regimen prior to infusion with donor BMC, to induce mixed hematopoietic chimerism and bilateral transplantation tolerance. Slavin et al., Science 193:1252 (1976); Slavin et al., J. Exp. Med. 146:34 (1977); Slavin et al., J. Exp. Med. 147:700 (1978); Slavin et al., J. Exp. Med. 147:963 (1978); Slavin S., Immunol. Today, 3:88 (1987); Slavin et al., Isr. J. Med Sci., 22:264 (1986). TLI is non-myeloablative and routinely given safely on an outpatient basis to transplant recipients and patients with Hodgkin's disease. Unfortunately, consistent induction of chimerism using TLI has required very high cumulative doses of radiation (3,400-4,400 cGy) which again would not be desirable for transplant recipients. TLI has significant advantages over TBI, especially in the clinical setting. TLI, which involves selective irradiation of the lymphoid compartment without exposing the whole body to ionizing irradiation, is well tolerated. In addition, TLI preserves intact a significant portion of the recipient's immunohematopoietic system, with resultant retained memory to recall antigens including infective agents. However, long courses of TLI can be time consuming and may be associated with short and long-term side effects that may not be suitable for routine clinical application.

WO 98/52582 teaches a method for inducing mixed hematopoietic chimerism and bilateral transplantation tolerance, which involves subsequent administrations of donor antigens and a lymphocytotoxic agent to the recipient. The underlying concept of this method is based on activating the T-cells lymphocytes that respond to non-syngeneic donor antigens and then selectively eliminating these lymphocytes by administration of a cytotoxic agent that kills proliferating cells. This non-myeloablative, donor-specific tolerogenic treatment resulted, according to the teachings of WO 98/52582, in conversion of a recipient to a hematopoietic mixed chimera with high levels of donor hematopoietic cells. The above method was practised using cyclophosphamide as the lymphocytotoxic agent. Cylophosphamide is an alkylating agent and therefore leads to death of rapidly dividing cells, which are highly susceptible to this kind of agents.

While WO 98/52582 teaches a method that is based on depletion of alloreactive cells through activation-induced cell death (AICD), which is effected by administration of cyclophosphamide, WO 98/52582 is silent with respect to other agents that may cause clonal deletion of alloreactive cells, via other mechanisms such as activation-induced apoptosis (AIA). WO 98/52582 is also silent with respect to the selectivity of cyclophosphamide in eliminating or inactivating the alloreactive lymphocytes exclusively, while enhancing the activity of other lymphocytes.

Tyrphostins are well known low molecular weight compounds, capable of modulating the activity of protein tyrosine kinase. Various classes of tyrphostins, as well as their activity as inhibitors of PDGF (platelet derived growth factor) receptor tyrosine kinase activity and hence as blockers of PDGF-dependent cell proliferation and their activity as other receptor tyrosine kinase inhibitors are disclosed in U.S. Pat. Nos. 5,196,446, 5,217,999, 5,302,606, 5,656,655, 5,700,822, 5,700,823, 5,712,395, 5,763,441, 5,773,746, 5,789,427, 5,792,771, 5,849,742, 5,932,580, 5,981,569, 5,990,141, 6,126,917, 6,331555, 6,358,951, 6,258,954 and 5,661,147, in WO 01/34607, WO 99/07701, WO 99/53924, WO 96/29331, WO 92/20642, WO 91/16892, WO 91/16305, WO 91/16051, and are further taught by Leonard et al. J. Org. Chem., 40: 356, 1975. Levitzki, A. Tyrphostins: tyrosine kinase blockers as novel antiproliferative agents and dissectors of signal transduction. FASEB J., 6: 3275-3282, 1992. Bilder et al. Tyrphostins inhibit PDGF induced DNA synthesis acid associated early events in smooth muscles. Am. J. Physiol., 260: C721-C730, 1991. Bryckaert et al. Inhibition of platelet-derived growth factor-induced mitogenesis and tyrosine kinase activity in cultured bone marrow fibroblasts by tyrphostins. Exp. Cell. Res., 199: 255-261, 1992. Kovalenko et al. Selective platelet-derived growth factor receptor kinase blockers reverse sis-transformation. Cancer Research, 54:6106-6114. Kovalenko et al. Phosphorylation site-specific inhibition of platelet-derived growth factor α-receptor autophosphorylation by the receptor blocking tyrphostin AG 1296. Biochemistry, 36:6260-6269. All the references cited supra are incorporated by reference as if fully set forth herein.

While conceiving the present invention, it was hypothesized that the high and efficient activity of tyrphostins as selective inhibitors of specific receptor tyrosine kinase activity and/or non-receptor tyrosine kinase activity could be utilized to exclusively eliminate alloreactive lymphocytes that are activated upon administration of donor antigens to a recipient, by eliminating the signal transduction pathways that are involved in the activation or proliferation of these lymphocytes or by enhancing the apoptosis of these cells. Hence, it was further hypothesized that subsequent administrations of donor antigens and of tyrphostins, would result in clonal deletion of the alloreactive cells and hence in induction of host-versus-graft and graft-versus-host unresponsiveness and consequently in bilateral transplantation tolerance.

### SUMMARY OF THE INVENTION

While reducing the present invention to practice, it was surprisingly found that administering a recipient with donor antigens in the presence of various tyrphostins resulted in efficient clonal-specific inactivation of the alloreactive lymphocytes. Moreover, it was found that such a treatment resulted in unaffected and, in some cases, even enhanced function of other T cell subsets.

Hence, the present invention provides a new method for inducing immune tolerance of a mammal to cells, tissue and/or organ allografts and xenografts. The present invention further provides a new method for inducing self-immune tolerance.

In one aspect, the present invention provides a method of inducing immune tolerance in a first mammal to antigens of a second, non-syngeneic (i.e., allogeneic or xenogeneic), mammal. The method comprising administering antigens from the second mammal to the first mammal and administering a non-myeloablative dose of one or more tyrphostin(s) to the first mammal, to selectively eliminate mammal lymphocytes responding to the antigens.

The method can further comprise, prior to, or concomitant with, administering the antigens from the second mammal, administering one or more immunosuppressive agent(s) to the first mammal, in a non-myeloablative regimen sufficient to decrease the functional T lymphocyte population of the first mammal.

The immunosuppressive agent(s) can include one or more of an immunosuppressive drug, an alkylating agent, ionizing radiation, or anti-leukocyte or anti-leukocyte function antibodies. It is particularly advantageous to use a short course of TLI (sTLI) as the immunosuppressive agent, for example 1-12, frequently 1-6, doses of 200 cGy/dose.

The antigens of the second mammal that are administered to the first mammal can include non-cellular antigens, cells, tissues and/or organs. For example, the antigens can include hematopoietic stem cells or other viable cells. If the antigens include hematopoietic stem cells, then the immunosuppressive regimen referenced above should decrease the T lymphocyte population of the first mammal to a level permitting at least transient survival of these cells. For example, the T lymphocyte population of the first mammal can be decreased by 90%, 95% or 99%. The first mammal can be an animal or a human, for example a human cancer patient. The second mammal can be allogeneic or xenogeneic to the first mammal.

In another aspect, the present invention provides a method of transplanting in a first mammal a graft derived from a second mammal, while minimizing graft rejection. The method comprises inducing immune tolerance in the first mammal to antigens of the second, non-syngeneic, mammal, as is described hereinabove, prior to the transplantation.

The graft that is most suitable for the transplantation method of this aspect of the invention can be an organ or a tissue that is not rich in immunocompetent lymphocytes (e.g., heart or kidney). However, the method can further comprise administering a preparation of stem cells of the second mammal to the first mammal with resultant engraftment of such cells in the first mammal. After administering the preparation of hematopoietic stem cells, the blood of the first mammal can contain 20% or more cells of the second mammal and the first mammal can be treated with allogeneic cell therapy. This involves infusing allogeneic lymphocytes from the second mammal into the first mammal. In yet another aspect, the present invention provides a method of transplanting a graft derived from a first mammal in a second mammal while reducing graft-versus-host disease by inducing immune tolerance in the first mammal to antigens of the second, non-syngeneic, mammal. The method comprising administering antigens from the second mammal to the first mammal, administering a non-myeloablative dose of one or more tyrphostin to the first mammal, to selectively eliminate mammal lymphocytes responding to the antigens, and transplanting the graft in the second mammal.

The graft that is transplanted by this method is preferably a graft rich in immunocompetent lymphocytes, such as bone marrow cells, small intestine and pancreatic islets, which, upon the method described hereinabove, becomes tolerogenic to the second mammal and therefore do not cause the graft-versus-host disease.

In still another aspect, the present invention provides a method of inducing bilateral immune tolerance in a first mammal and a second, non-syngeneic, second mammal. The method comprises inducing immune tolerance in a first mammal to antigens of a second, non-syngeneic, mammal and inducing immune tolerance in the second mammal to antigens of the first mammal, using the method described hereinabove for inducing immune tolerance in both the first and second mammals.

In an additional aspect, the present invention provides a method of transplanting a graft derived from a first mammal in a second mammal, while reducing both graft rejection and graft-versus-host disease. The transplantation is performed, according to this aspect of the present invention, following induction of bilateral immune tolerance in the first mammal and in the second, non-syngeneic, second mammal, as is described hereinabove.

This method enables transplantation of a graft that is rich in immunocompetent lymphocytes, as is detailed hereinabove, by inducing mixed chimerism in both the graft-donor and graft-recipient.

Using the methods described hereinabove, a mixed, non-human mammal/human chimera can be produced, using a method that comprises inducing immune tolerance of a non-human mammal to human antigens and thereafter administering a preparation of hematopoietic stem cells from the human to the mammal.

The non-human mammals can be, for example, a rodent or a pig, and hence, the present invention provides a rodent, a pig or other non-human mammal, which is stably engrafted with human hematopoietic stem cells. As such, the non-human mammal constitutes a hematopoietic mixed chimera.

Similarly, a first non-human mammal/second non-human mammal hematopoietic mixed chimera can be produced.

In another aspect, the present invention provides a method for inducing self-immune tolerance, by administering to a mammal specific antigens, such as factor 8 protein or antigens involved in an autoimmune disease, and subsequently administering to the mammal a non-myeloablative dose of one or more tyrphostin(s), to selectively eliminate mammal lymphocytes responding to these specific antigens.

The induction of immune tolerance according to the present invention is effected by administering one or more tyrphostin(s). The tyrphostin(s) eliminate lymphocytes responding to the administered antigens.

Hence, according to another aspect of the present invention, there is provided a packaged pharmaceutical composition comprising, as an active ingredient, an effective amount of one or more tyrphostin(s) and a pharmaceutically acceptable carrier. The pharmaceutical composition is packaged in a package and is identified in print associated with the package for use in an immune tolerance application. The immune tolerance application can therefore be any of the methods described hereinabove.

Various tyrphostins that belong to various families can be used in the methods and composition of the present invention. Hence, tyrphostin(s) of the quinoxaline family, the quinazoline family, the cyano-substituted acrylamide family, the cyano-substituted thioacrylamide family, the acrylonitrile family, the phenyl-substituted acrylonitrile family, the substituted aniline family, the benzoxazolone family, the tricyclic pyridone family and the tetracyclic pyridone family, can be utilized by the present invention.

As a result, the present invention further provides a method of determining, both in vitro and in vivo, an activity of a tyrphostin in selective elimination of lymphocytes of a first mammal, that are responding to antigens of a second, non-syngeneic, mammal.

This method comprises stimulating hematopoietic cells of the first mammal with first antigens of the second mammal in a presence of and without the tyrphostin, and thereafter exposing the hematopoietic cells of the first mammal to second antigens of the second mammal without the tyrphostin and measuring a response of the blood mononuclear cells of the first mammal to the antigens of the second mammal.

The present invention further provides a method of determining an optimal concentration of a tyrphostin for selective elimination of lymphocytes of a first mammal, that are responding to antigens of a second, non-syngeneic, mammal. This method comprises stimulating hematopoietic cells of the first mammal in the presence of different concentrations of the tyrphostin, as is described hereinabove, and thereafter exposing these hematopoietic cells to second antigens, as is described hereinabove, and measuring their response.

The term “non-myeloablative” as used herein includes any therapy that does not eliminate substantially all hematopoietic cells of an administered mammal.

“Transplantation” as used herein refers to transplantation of any donor-derived material including cells, tissues and organs. The cells may be hematopoietic or non-hematopoietic.

“Antigens” as used herein refers to any material that elicits an immune response, including non-cellular antigens, cells, tissues or organs. Stem cells are particularly useful as antigens.

The term “cancer” as used herein includes all pathological conditions involving malignant cells; this can include “solid” tumours arising in solid tissues or organs as well as hematopoietic tumors such as leukemias and lymphomas.

The term “immune tolerance” as used herein refers to tolerance of one mammal to a material derived from another mammal.

In one particular, immune tolerance is used herein to describe donor-specific tolerance.

In another particular, the immune tolerance is used herein to describe self-antigens tolerance.

The term “donor-specific tolerance” as used herein refers to tolerance of the recipient to donor-derived material.

Induction of donor-specific tolerance across strong major histocompatibility complex (MHC) and minor histocompatibility complex (MiHC) barriers, as well as across species barriers (xenogeneic tolerance) may be achieved in mammalian recipients using the tolerogenic treatment described herein. Induction of donor-specific transplantation tolerance while avoiding the need for maintenance immunosuppressive treatment is a highly desirable goal in clinical transplantation.

The non-myeloablative tolerogenic treatment described herein induces a state of long-lasting donor-specific tolerance to a wide variety of donor-derived materials. Such an approach is attractive for allogeneic and xenogeneic transplantation of cells, tissues and organs in clinical settings, since all the steps of the protocol are well tolerated and relatively safe. Since there is no need to eradicate the entire recipient immunohematopoietic system during the course of the procedure, the recipients retain immune memory and are in a better position to resist graft-versus-host disease on the one hand and infectious complications on the other. This can be of crucial importance in clinical practice. The protocols for inducing donor-specific tolerance may be delivered, at least in part, as outpatient procedures.

The non-myeloablative tolerogenic treatment described herein further encompasses a self-immune tolerance which can be used, for example, in the treatment of a wide variety of autoimmune diseases and/or diseases having an autoimmune component.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the patent specification, including definitions, will control. In addition, the materials, methods, and examples are illustrative only and not intended to be limiting.

Other features and advantages of the invention will be apparent from the following detailed description, and front the claims.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1(a-i) presents the chemical structures of exemplary tyrphostin compounds (Tyr 1-Tyr 37) useable in accordance with the teachings of the present invention;

FIG. 2 presents plots demonstrating the effect of various concentrations of three tyrphostins (Tyr 1, Tyr 2 and Tyr 5) on primary MLR (mixed lymphocytes reaction). The percent response was calculated from ³[H]Tdr uptake; and

FIG. 3 is a schematic description of an exemplary skin grafting procedure according to the present invention.

### DESCRIPTION OF THE PREFERRED EMBODIMENTS

The present invention is of novel, non-myeloablative tolerogenic treatments, which induce stable and donor-specific tolerance to non-syngeneic transplants (i.e., transplants of cells, tissues or organs which are not genetically identical to the recipient). Specifically, the tolerogenic treatments of the present invention result in induction of immune tolerance of one mammal to antigens of another, non-syngeneic, mammal, and hence can be utilized to minimize graft rejection and/or to reduce graft-versus-host disease (GVHD) and other graft-related diseases. The tolerogenic treatments of the present invention can be used to induce immune tolerance to any desirable antigen and hence can be utilized in the treatment of various disease such as, for example, autoimmune diseases or diseases having an autoimmune component.

The principles and operation of the tolerogenic treatments according to the present invention may be better understood with reference to the drawings and accompanying descriptions.

Before explaining at least one embodiment of the invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting.

Broadly stated, the tolerogenic treatments of the present invention are based on a method of inducing immune tolerance in a first mammal to antigens of a second, non-syngeneic, mammal. The method is effected by the following procedures:

(a) infusion of antigens from the second mammal to the first mammal; and

(b) administering a non-myeloablative dose of one or more tyrphostin(s) to the first mammal, to selectively eliminate mammal lymphocytes responding to the antigens.

For the purpose of convenience, the phrase “a first mammal” is also referred to hereinafter, with respect to this aspect of the present invention, as “a donor”. The phrase “a second mammal” is therefore referred to hereinafter as “a recipient”.

Prior to or concomitant with the antigens infusion, one or more immunosuppressive agent(s) are preferably administered to the first mammal, in a non-myeloablative regimen sufficient to decrease, but not eliminate, the functional T lymphocyte population thereof. The functional T lymphocyte population includes, for example, the antigen-responding T lymphocytes and other lymphocyte subsets such as B cells or alloreactive NK cells. The administration of an immunosuppressive agent prior to or concomitant with the tyrphostin(s), typically results in synergistic elimination of the antigens-responding lymphocytes.

Examples of immunosuppressive agents useful in the context of the present invention include, without limitation, immunosuppressive drugs such as methotrexate, cyclosporine, sirolimus (rapamune), tacrolimus (prograf) and fludarabine (FLU); alkylating agents such as Cy, melphalan, thiotepa and busulfan; polyclonal and monoclonal anti-thymocyte globulin (ATG) and anti-lymphocyte globulin (ALG); antibodies radiolabelled with radioactive isotopes; and ionizing radiation such as TLI and TBI. Due to its non-selective effects on all of the recipient's hematopoietic cells and its severe immediate and long-term side effects, TBI is not preferred. If TBI is used, it should be at a dose level that causes no severe or irreversible pancytopenia. The non-myeloablative regimen advantageously is a short and well-tolerated course of TLI (sTLI) which may cause a major reduction in the number and/or function of recipient T lymphocytes in all lymphoid organs. As discussed hereinabove, it has been discovered that sTLI can effectively induce unresponsiveness to donor antigens at relatively low cumulative radiation doses.

The sTLI immunosuppressive regimen may comprise, for example, 1 to 12 daily fractions of 200 cGy/each. The fractions' number depends on the procedures that follows and their potential. For example, if the immune tolerance is induced in order to minimize host-versus-graft rejection, the sTLI immunosuppressive regimen depends on the host-versus-graft potential. If the immune tolerance is induced prior to engraftment of donor-derived stem cell, the sTLI immunosuppressive regimen depends on the T lymphocyte content in the administered stem cell preparation. Stem cell preparations rich in T lymphocytes may require only 1-3 sTLI fractions, or may not require immunosuppression at all (zero sTLI fractions). Transplantation of T cell-depleted stem cell preparations or stem cell preparations with low levels of T lymphocytes, however, may require the use of 4-12 fractions. The sTLI regimen is highly advantageous as it causes only a transient reduction in the number of recipient T lymphocytes and it is clinically feasible on an outpatient basis. Furthermore, there are no anticipated severe side effects since a routine cumulative dose of TLI used clinically for lymphoma patients consists of 4,400 cGy.

Preferably, the immunosuppressive agent transiently decreases the recipient functional T lymphocyte population by at least about 90%. More preferably, the non-myeloablative regimen transiently decreases the recipient functional T lymphocyte population by at least about 95%, and most preferably, by at least about 99%. Reductions of less than 90% of the lymphocytes are also within the scope of this invention, provided that transient survival of donor antigens, in the procedure that follows, is possible.

In some donor/recipient combinations, tolerance to donor antigens may be inducible without the necessity of administering immunosuppressive agents.

Following the administration of the immunosuppressive agent, or as a first step of the induction of immune tolerance of the present invention, antigens from a non-syngeneic donor are administered to the recipient mammal in order to stimulate and cause proliferation of donor-specific T lymphocytes of the recipient. Hence, the donor antigens may be administered to a recipient that is administered with the non-myeloablative immunosuppressive regimen described above or to a non-immunosuppressed recipient.

The antigens of the first mammal (the donor antigens) include, without limitation, non-cellular antigens, cells, organs, tissues, either live or killed, or extracts thereof, or even anti-idiotypic antibodies that mimic donor antigens. In general, any donor antigens that elicit an immune response in the recipient are within the scope of this invention. Any source of donor antigens from a non-syngeneic donor can be used, and the non-syngeneic donor can be allogeneic or xenogeneic to the recipient, as these terms are defined hereinabove.

The infusion of donor antigens should comprise donor antigenic determinants for which tolerance is desired. For example, if it is desired, following the induction of the immune tolerance, to transplant into the recipient donor-derived material bearing only class I histocompatibility antigens, it may be necessary to eliminate only class I-reactive recipient T lymphocytes. This could be accomplished by infusing donor antigens bearing only class I antigenic determinants. On the other hand, additional donor antigenic determinants may be present in the infusion even though recipient tolerance to these additional antigenic determinants may not be necessary.

Thus, elimination of class I-reactive and class II-reactive recipient T lymphocytes by infusion of donor antigens bearing class I and class II antigenic determinants may be performed even if the later transplanted donor material bears only Class I antigenic determinants.

The donor antigens are preferably viable hematopoietic stem cells from a non-syngeneic donor. The donor hematopoietic stem cells are generally not T cell depleted, although use of T cell depleted donor hematopoietic stem cells in this procedure is also within the scope of this invention. Donor hematopoietic stem cells may be obtained, for example, by direct extraction from the bone marrow or from the peripheral circulation following mobilization from the bone marrow. The latter can be accomplished by treatment of the donor with granulocyte colony stimulating factor (G-CSF) or other appropriate factors that induce mobilization of stem cells from the bone marrow into the peripheral circulation. The mobilized stem cells can be collected from peripheral blood by any appropriate cell pheresis technique, for example through use of a commercially available blood collection device, as exemplified by the CS 3000 Plus blood cell collection device marketed by the Fenwal Division of Baxter Healthcare Corporation, or use of an equivalent device marketed by Kobe Spectra and other companies, preferably in a closed system. Methods for performing a pheresis with the CS 3000 Plus machine are described in Williams et al. Bone Marrow Transplantation 5: 129-133 (1990) and Hillyer et al., Transfusion 33: 316-321 (1993). Alternative sources of stem cells include neonatal stem cells (e.g., cord blood stem cells) and fetal stem cells (e.g., fetal liver of yolk sac cells). Stem cells that have been expanded in vitro with a mixture of hematopoietic cytokines also may be used. Other useful stem cell preparations include stem cells that have been transduced with genes encoding donor-type MHC class I or class II molecules, as well as stem cell preparations containing stem cells and/or T cells transduced with herpes simplex thymidine kinase or other “suicide” genes to render the mature T cells sensitive to ganciclovir or other appropriate drugs in the event of severe GVHD.

Following the infusion of donor antigens, one or more tyrphostin(s) are administered to the recipient mammal (the first mammal), to selectively eliminate the proliferating donor-specific recipient T lymphocytes. The “elimination”, as used herein, includes inactivation of the proliferating T lymphocyte in the recipient, preferably by activation-induced apoptosis (AIA) and/or by activation-induced cell death (AICD).

As is discussed herein, tyrphostins include various classes of compounds that are capable of modulating the activity of protein tyrosine kinase.

Receptor tyrosine kinases (RTKs) comprise a large family of transmembrane receptors for polypeptide growth factors with diverse biological activities. The intrinsic function of RTKs is activated upon ligand binding, which results in phosphorylation of the receptor and multiple cellular substrates, and subsequently in a variety of cellular responses. RTKs, as well as, more generally, protein tyrosine kinases, play an important role in the control of cell growth and differentiation. Aberrant expression or mutations in the RTKs have been shown to lead to either uncontrolled cell proliferation (e.g. malignant tumor growth) or to defects in key developmental processes.

Inhibition of the activity of protein tyrosine kinase may therefore lead, inter alia, to apoptosis and hence to inactivation of proliferating cells, and therefore the use of RTKs inhibitors in eliminating proliferating lymphocytes is highly beneficial.

Various families of tyrphostins that are known in the art are usable in the context of the present invention. Hence, the tyrphostins of the present invention include, without limitation, various derivatives of quinoxalines, quinazolines, cyano-substituted acrylamides, cyano-substituted thioacrylamides, acrylonitriles, phenyl-substituted acrylonitriles, substituted acrylonitriles, phenyl-substituted acrylonitriles, substituted anilines, benzoxazolones, tricyclic pyridones and tetracyclic pyridones.

Representative examples of tyrphostins that can eliminate responding lymphocytes, by AIA and/or by AICD, include, without limitation: N-benzyl-2-cyano-3-(3,4-dihydroxyphenyl)-acrylamide, N-benzyl-2-cyano-3-[3,4-dihydroxy-5-(3-phenylpropylsulfanylmethyl)-phenyl]-acrylamide, 4-amino-1-t-butyl-3-(p-toluyl)pyrazolo[3,4-d]pyrimidine, 4-amino-1-t-butyl-3-(p-chlorophenyl)pyrazolo[3,4-d]pyrimidine, 2-cyano-3-(3,4-dihydroxyphenyl)-N-(4-phenylbutyl)-acrylamide, 2-cyano-3-(3,4-dihydroxy-5-phenethylsulfanylmethyl-phenyl)-N-(4-phenylbutyl)-acrylamide, 2-(3-hydroxy-4-nitro-benzylidene)malononitrile, 4-(6,7-dimethoxyquinazoline-4-yl-amino)-benzamide, 4-(6,7-dimethoxyquinazoline-4-yl-amino)-phenol, 4-amino-1-t-butyl-3-(2-thiophene)pyrazolo[3,4-d]pyrimidine, 3-(3,5-dimethyl-H-pyrrol-2-yl-methylene)-2-oxo-2,3-dihydro-1H-indole-5-sulfonic acid dimethylamide, 4-amino-1-t-butyl-3-(3,4-dimethoxyphenyl)pyrazolo[3,4-d]pyrimidine, 2-cyano-3-(3,5-di-t-butyl-4-hydroxy phenyl)-thioacrylamide, 3-amino-5-[1-cyano-2-(1H-indol-3-yl)vinyl]-1H-pyrazole-4-carbonitrile, N-benzyl-2-cyano-3-(3,4-dihydroxy phenyl)-thioacrylamide, 2,3-dicyano-6-phenyl-pyridazine, 2-cyano-3-(3,4-dihydroxy-5-iodophenyl)-N-(4-phenyl propyl)-acrylamide, 2-(4-methoxy-benzylidene)malononitrile, and 2-cyano-3-(3,4-dihydroxyphenyl)-thioacrylamide.

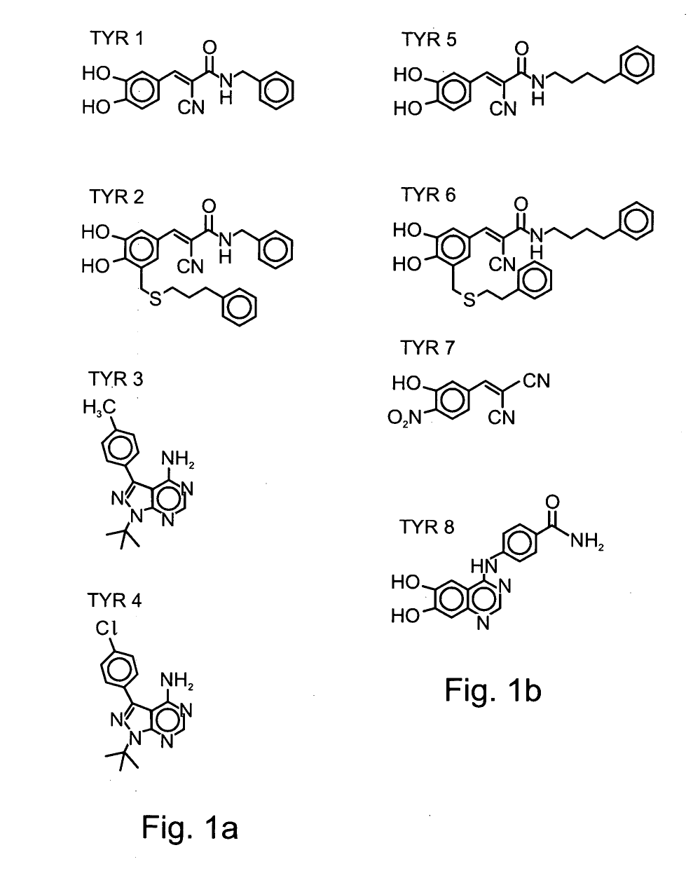

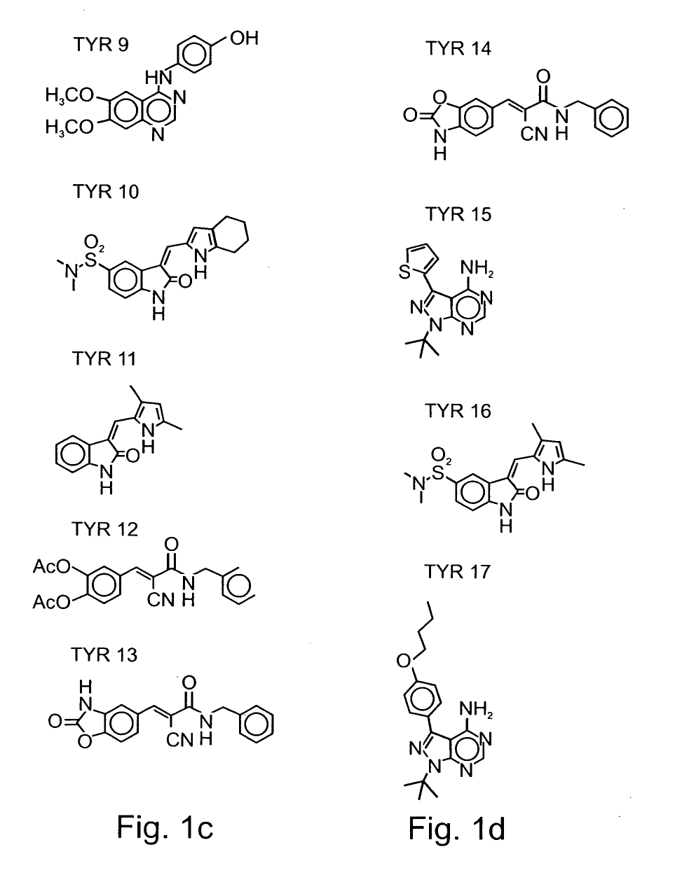

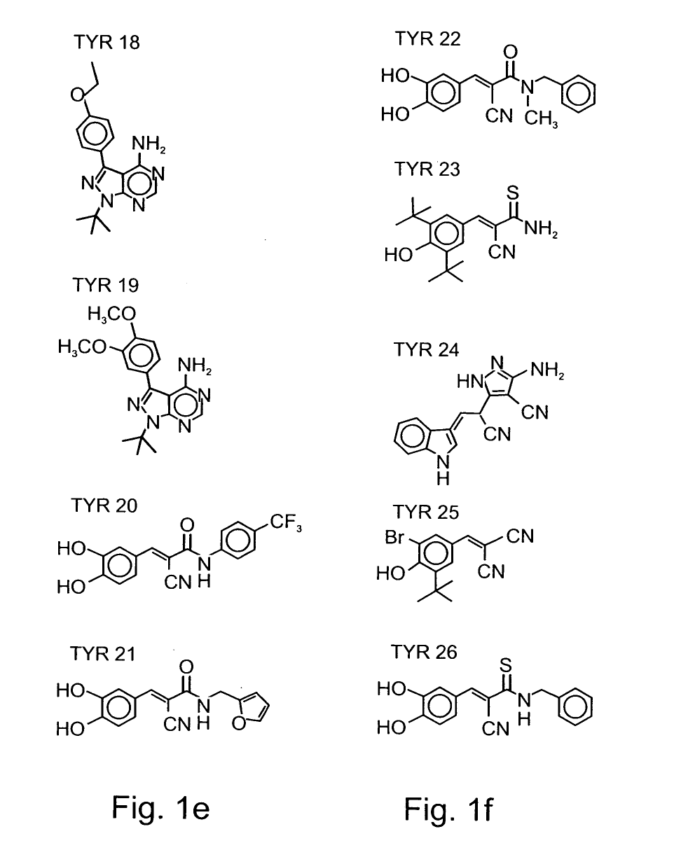

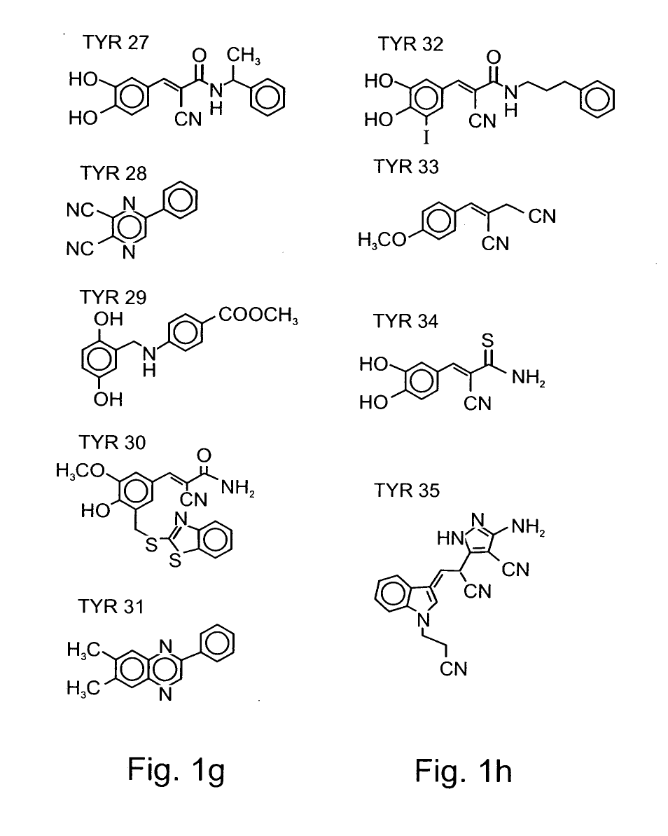

The chemical formulas of these tyrphostins and other representative examples of tyrphostins that are usable in the context of the present invention are presented in FIG. 1(a-i).

However, other tyrphostins of other families can also be used in the context of the present invention.

Following the administration of one or more tyrphostin(s), with or without the administration of an immunosuppressive agent, a donor-specific tolerance is induced in the first, recipient mammal. This non-myeloablative, donor-specific tolerogenic treatment results in conversion of a recipient to a hematopoietic mixed chimera.

The immune tolerance induced by the method described hereinabove enables to perform subsequent transplantation of a graft derived from one mammal in another, non-syngeneic, mammal, while minimizing graft rejection and/or GVHD, as is detailed hereinbelow.

Typically, the mammalian recipients are human patients, although a recipient of the tolerogenic treatment may be any mammal. Non-syngeneic transplantation can include allogeneic as well as xenogeneic transplantation of organs, tissues or cells. Hence, hematopoietic stem cells and other donor antigens may be derived from allogeneic or xenogeneic sources.

Human patients for which the tolerogenic treatment is appropriate include, without limitation, those with loss of organ or tissue function including loss of metabolic function such as in type I diabetes; patients with enzyme deficiencies caused by inborn genetic diseases such as Gaucher's disease, metachromatic leukodystrophy and Hurler's Syndrome; patients with autoimmune disorders such as multiple sclerosis, lupus erythematosus and rheumatoid arthritis; patients with genetic diseases treatable by stem cell transplantation such as beta thalassemia major, side cell anemia and cancer patients having hematologic malignancies and certain metastatic solid tumors. Patients suffering from heart, liver or kidney failure, for example, are excellent candidates for conditioning with the tolerogenic treatment prior to transplantation with the appropriate organ. Patients requiring a skin or bone graft may also be subjected to the tolerogenic treatment prior to grafting. Cancer patients receiving the tolerogenic treatment can include patients suffering from any malignancy, either solid tumors such as renal cell cancer, breast cancer or hematopoietic malignancies including acute and chronic leukaemia, lymphoma, and myelodysplastic and myeloproliferative disorders.

Hence, in one method, the induction of the immune tolerance of the present invention is followed by transplanting a graft that is derived from the second, donor mammal in the first, recipient mammal, while minimizing the graft rejection.

This method is particularly effective in cases where the graft is an organ, a tissue or cells that is/are not rich in immunocompetent lymphocytes or in cases where the graft is derived from a partially matched donor. In such cases, the elimination of the responding lymphocytes may be sufficient to induce minimized graft rejection without increasing the proportion of chimerism, as antigens shed by the graft itself may provide sufficient mandatory for maintenance of tolerance. Examples of organs that are suitable grafts in this method include, without limitation, heart, lung, liver, kidney and pancreas.

However, in order to ensure an acceptable state of stable, mixed chimerism with relatively high numbers of circulating donor cells, and hence to assure a more robust transplantation tolerance, donor hematopoietic stem cells can be administered to the recipient prior to the transplantation. This infusion of donor stem cells is derived from the same donor, or from a donor genetically identical to that providing the antigens. Hematopoietic stem cells from bone marrow, from mobilized peripheral blood populations, cryo-preserved cord blood, hematopoietic stem cells expanded in vitro by hematopoietic growth factors or other stem cell preparations as described above, may be used. The number of the administered stem cells can vary depending on the T cell content of the stem cell preparation. If the preparation is not T cell-depleted, then relatively small numbers of stem cells generally are administered. If the stem cell preparation is T cell-depleted, then larger numbers of stem cells can be administered since there is no risk of GVHD, as is detailed hereinbelow. In general, since the elimination of donor-responding lymphocytes in substantially enhanced by the tyrphostin(s), administration of lower number of donor stem cells may be sufficient to ensure safe transplantation of donor hematopoietic cells and for induction of unresponsiveness and consequently specific transplantation tolerance.

The donor hematopoietic stem cells of the second infusion mayor may not be T cell depleted, depending on the immunologic disparity between the donor and recipient, the presence and intensity of the immunosuppression given prior to, or concomitant with, administering the stimulating antigens and the degree of chimerism desirable in view of the immunogenicity of the graft. When higher fractions of TLI (4-12), or other immunosuppressive agents providing equivalent immunosuppression, are used in the immunosuppressive regimen described hereinabove, the second infusion of donor hematopoietic stem cells typically is T cell depleted to control for GVHD. T cells present in the second infusion of donor hematopoietic stem cells can eliminate residual hematopoietic stem cells and residual T cells of the host. Therefore, when little immunosuppression is involved (for example, 1-3 fractions of sTLI), or when immunosuppression is eliminated altogether, the infusion of donor hematopoietic stem cells without T cell depletion is essential to displace all residual host cells. Following adequate immunosuppression (for example, more than 6 fractions of TLI) or as a result of using a tyrphostin or a tyrphostins combination with enhanced activity, the presence of T cells in the second stem cell infusion may not be required and hence purified stem cells or T cell depleted stem cells may be used with no risk of GVHD. If not T cell depleted, the donor stem cells can be infused in graded increments over a period of weeks or several months, while monitoring for signs of GVHD.

As a result of the infusion of the preparation of hematopoietic stem cells, the blood of the recipient preferably includes more than 20% of the donor and, more preferably, more than 50% cells of the donor and hence a stable hematopoietic mixed chimerism is achieved.

The administration of the preparation of hematopoietic stem cells provides a platform for subsequent allogeneic cell therapy with donor lymphocyte infusions in cancer patients and in other patients with malignant and non-malignant diseases requiring bone marrow transplantation, since donor cells accepted by a tolerant recipient may induce graft-versus-leukemia (GVL) or graft-versus-tumor (GVT) effects. Such non-malignant diseases include without limitation aplastic anemia, genetic diseases resulting in enzyme deficiencies, and diseases caused by deficiencies in well-defined products of hematopoietic stem cells, such as osteoclast deficiency in infantile osteopetrosis and deficiencies in B cells and T cells in congenital and acquired immune-deficiency syndromes. Allogeneic cell therapy is described, for example, in WO 95/24910 and WO 96/37208.

In allogeneic cell therapy, an anti-tumor or other anti-recipient hematopoietic cell effect is achieved by administering allogeneic peripheral blood lymphocytes to the recipient, either alone or in combination with a T cell activator. Alternatively, allogeneic peripheral blood lymphocytes are “pre-activated” in vitro by a T cell activator such as interleukin-2 (IL-2) and then administered either alone or in combination with the same or different T cell activator. Optionally, immune T cells may be used to achieve a more effective elimination of tumor cells or other hematopoietic cells of the recipient. Preferably, one or more infusions of about 105 to about 109 cells/kg of allogeneic peripheral blood lymphocytes, including well-defined lymphocyte subsets, are administered. When preceded by the tolerogenic treatment described hereinabove, these infusions of allogeneic lymphocytes are carried out with a much reduced chance of rejection of the anti-cancer effector cells, which need to become engrafted in the recipient. In addition, the risk of GVHD is reduced or eliminated by residual hematopoietic cells of the recipient and, if necessary, relatively late infusion of donor lymphocytes. The risk of GVHD can be further reduced by using immune, rather than naive, donor lymphocytes or cytotoxic lymphocytes generated in vitro, against well defined host targets such as melanoma cells, lymphoma cells (caused by Ebstein Bar virus), hepatoma cells (caused by hepatitis B virus) and cytomegalovirus.

The allogeneic cell therapy can be valuable not only in the context of cancer and other diseases, but also when it is desired to adoptively transfer immunity to infectious agents from the donor to the recipient. Thus, if a donor used in the induction of immune tolerance described hereinabove is immune to an infectious agent, this immunity can be transferred to a recipient by infusing lymphocytes from the donor to the recipient following completion of the tolerogenic treatment. Alternatively, the infusion of the stem cell preparation can itself provide the adoptive transfer of immunity, since stem cell preparations typically contain immunocompetent lymphocytes, which have an immune capacity that is identical to the donor. Hence, the infused stem cells can be further used for immune reconstitution of the recipient, for facilitating immune reconstitution or for re-establishing an immune system in the recipient. These features are beneficial, for example, in the treatment of congenital or acquired immune deficiency such as the treatment of patients with AIDS.

Although a significant number of functional T lymphocyte population of the recipient (first mammal) remains in the recipient after the non-myeloablative regimen described above, engraftment of the donor cells following the administration of the hematopoietic preparation can occur. This feature is enabled due to the elimination of the donor-reactive recipient T lymphocytes and since donor-derived T lymphocytes and/or stem cells present in the subsequent infusion or infusions) may act as “veto” cells to produce a veto effect. “Veto cells”, as used herein, include T lymphocytes, especially CD8+ T cells, that result in down regulation, rather than stimulation, of other T lymphocytes. Veto effects may be induced by other proliferating hematopoietic cells including T cell-depleted stem cells that are poorly immunogenic but that can veto recipient T cells. In the veto effect, recipient-originating T lymphocytes are down regulated by donor-derived veto cells, including stem cells and/or lymphocytes. Other replicating donor-derived cells, or even non-cellular antigens, can also veto recipient alloreactive or xenoreactive T cells if provided repeatedly and in relatively high concentrations. Similarly, immunocompetent T cells present in the donor infusion may be down regulated by veto cells of recipient origin. Thus, tolerance of graft-vs-host and host-vs-graft may occur simultaneously due to a balanced equilibrium between veto cells of recipient and donor origin on the one hand and the degree of immunogenicity and alloreactivity of the graft on the other.

Although the transplantation method described hereinabove provide immune tolerance of both host-versus-graft and graft-versus-host, as is discussed hereinabove, the above method does not secure elimination of GVHD. In order to ensure total prevention of GVHD, donor hematopoietic cells must be T cell depleted in vitro or in vivo prior to transplantation of a graft that is rich in hematopoietic cells.

Hence, successful reduction of GVHD can be achieved by elimination of alloreactive T cells of the donor in vivo, prior to transplanting its organs in the mammalian recipient. Such elimination is achieved, according to another method of the present invention, using the tolerogenic treatment of the present invention.

This method, which is aimed at reducing GVHD, is based on inducing immune tolerance as described hereinabove and is effected by administering antigens from the second mammal to the first mammal, administering a non-myeloablative dose of one or more tyrphostin to the first mammal, to selectively eliminate mammal lymphocytes responding to the antigens, and transplanting the graft in the second mammal.

The graft that is transplanted by this method is preferably a graft rich in immunocompetent lymphocytes, such as bone marrow cells, small intestines or other small organ that is rich in lymphocytes, which, as is discussed in detail hereinabove, typically results in the development of GVHD.

As opposed to the organ transplantation described hereinabove, in this method the graft is transplanted in the second mammal while the immune tolerance is induced in the first mammal, such that the first mammal is tolerant to antigens of the second mammal.

Hence, according to this method, the tolerogenic treatment results in induction of immune tolerance of the donor graft to antigens of the recipient and therefore results in substantial reduction of graft-versus-host or other graft-related diseases, as is detailed hereinabove, upon transplanting a graft that is rich in lymphocytes.

According to another aspect, the method of inducing immune tolerance of the present invention can be utilized to induce bilateral immune tolerance in a first mammal and in a second, non-syngeneic, mammal. The method is effected by inducing immune tolerance of a first mammal to antigens of a second mammal, according to the method described hereinabove, and similarly, inducing immune tolerance of the second mammal to antigens of the first mammal. This method can further include administration of one or more immunosuppressive agent(s), prior to the administration of the antigens, to the first and/or the second mammals.

By performing the tolerogenic treatment of the present invention in both the graft-donor mammal and the graft-recipient mammal, mixed chimerism is achieved in both the graft-donor and graft-recipient mammals, which results in bilateral immune tolerance.

The resultant bilateral immune tolerance enables to perform transplantation of various organs, tissues or cells, which are either rich or poor in lymphocytes, of one mammal in the other mammal, while minimizing graft rejection and reducing GVHD simultaneously.

The tolerant mixed hematopoietic chimeras generated by the tolerogenic treatment described herein remain immunocompetent to third party antigens.

As is further demonstrated in the Examples section that follows, cells that were treated by the method of the present invention did not show reduced immunoresponse neither to third party grafts nor to various mitogens. Thus, the immune tolerance induction of the present invention neither eliminates nor impairs normal reactivity by the recipient immune system retained in the mixed chimera. This is an important advantage of the tolerogenic treatment of the present invention, since recipients are not immunocompromised due to transient loss of all recipient-derived immune cells, which is otherwise unavoidable when chimeras are comprised of 100% donor cells following TBI. A patient who retains a recipient-derived immune apparatus with memory cells is in a better position to resist primary and secondary infections. This retained resistance to intercurrent infections, particularly to viral agents infecting recipient target cells, is of crucial importance. This is because the donor hematopoietic cells may be MHC disparate and, therefore, incapable of providing immune protection against virally-infected recipient tissues.

The tolerant mixed hematopoietic chimerism generated by the tolerogenic treatment described herein can be converted to 100% donor chimerism, whenever it is desirable. This can be easily obtained by donor lymphocyte infusion at a later stage, when the risk of GVHD is reduced, especially when graded increments of donor lymphocytes is used, as is discussed herein above.

The above-described tolerogenic treatment may be employed to induce transplantation tolerance across allogeneic and xenogeneic barriers.

Thus, in another aspect, the present invention provides a method of producing a hematopoietic mixed non-human mammal/human chimera.

In one embodiment, the method is effected by inducing immune tolerance of a non-human mammal to antigens originating from a human donor, using the non-myeloablative tolerogenic treatment described herein. That is, the non-human mammal functions as the “recipient mammal” in the protocols described above, and a human being is the “donor”. For example, a rodent can be tolerized to human cells, tissues and organs by employing the disclosed tolerogenic protocol, followed by infusion of human hematopoietic stem cells, to produce a mixed chimera rodent permanently engrafted with human hematopoietic cells. It is known that such hematopoietic engraftment is possible even between disparate species. For example, it has been demonstrated that human hematopoietic cells can engraft in mice. See, for example, Marcus et al., Blood 86: 398-406 (1995). In those cases where survival and functioning of human hematopoietic cells is less than optimal in non-human mammalian hosts, it is possible to provide the recipient mammal with human hematopoietic cytokines in order to ensure engraftment of the human cells. In another embodiment, the method is similarly effected by inducing immune tolerance of a human to antigens originating from a non-human mammal donor.

There are numerous uses for such mixed animal/human chimera. For example, in cases where the recipient mammals have been tolerized to the human donor, it is possible for human tissues, e.g., tumors or HIV-infected hematopoietic cells, to be transplanted into and accepted by these rodents in order to produce rodent models of human disease. Thus, these non-human mammal/human chimeras may be used to study biological phenomena related to human disease, including testing of new drugs, as well as for using secondary hosts for in vivo expansion of human cells or tissues from hematopoietic stem cells, mesangial stem cells or embryonic stem cells, respectively.

Production of hematopoietic mixed non-human mammal/human chimeras is of even greater significance for those non-human mammalian species targeted as potential sources of cells, tissues and organs for transplantation into human patients. For example, it is widely recognized that pigs are a potential useful source of tissues and organs for transplantation into humans. Such porcine materials are subject to an immediate, “hyperacute” rejection response when transplanted into human patients, as well as to longer-term immune-mediated rejection by the human recipient. Pigs are being genetically engineered or otherwise treated to protect tissues and organs of such pigs from being hyperacutely rejected when transplanted into a human patient. This can be accomplished, for example, by providing the pigs human genes encoding human complement regulatory proteins, or by “knocking out” the genes responsible for production of pig antigens recognized by performed xenoantibodies present in all humans. See, for example, PCT/US96/15255 and PCT/IB95/00088.

A “two-way” variation of the tolerogenic treatment of the present invention can be applied to such genetically engineered pigs as well as to other donor mammals, to allow for ready transplantation of xenogeneic donor cells, tissues and organs into humans. For example, in a preliminary tolerization procedure, a human patient can function as an initial “donor” to provide antigens to a “recipient” pig, using the tolerogenic treatment described above. Following administration of human hematopoietic stem cells, the pig is transformed into a pig/human hematopoietic mixed chimera, with the pig's hematopoietic cells being tolerized to the human patient's cells, tissues and organs. Following this, the roles of the human patient and pig are reversed, with the pig becoming the donor and the human patient becoming the recipient in the tolerogenic treatment. That is, the pig's hematopoietic cells, with T cells tolerant of the human patient, may be used in the method of inducing immune tolerance for transformation of the human patient into a human/pig hematopoietic mixed chimera. The human patient is then able to accept cells, tissues and organs from the pig, for the reasons discussed above. The crucial advantage is that all of this can be accomplished while avoiding the risk of xenogeneic GVHD engendered by immunocompetent T cells of the pig, since the pig's T cells were made tolerant to the patient in the preliminary tolerization procedure. Thus, assuming the hyperacute rejection response can be overcome in other ways (e.g., genetic engineering of the animal providing the transplanted material), the present invention allows for xenogeneic transplantation of cells, tissues and organs into humans without the need for long-term immunosuppression.

The principles of the tolerogenic treatment described hereinabove can be further practiced with any desirable antigens. In particular, the tolerogenic treatment of the present invention can be further used for inducing immune tolerance to any antigens that involve undesirable immunoresponse. Such antigens include, without limitation, factor 8 proteins, autoimmune disease-related antigens and antigens associated with an autoimmune component of other diseases.

Thus, according to another aspect of the present invention, there is provided a method of inducing immune tolerance to specific antigens. This method is effected by administering to a patient is need specific antigens and subsequently administering a non-myeloablative dose of one or more tyrphostin(s), to selectively eliminate the lymphocytes responding to these specific antigens. The specific antigens can be for example self antigens. The efficacy of this treatment can be further enhanced when the administered tyrphostin(s) further induce enhancement of the immune system, as is discussed in detail hereinbelow.

All the tolerogenic treatments of the present invention involve the administration of one or more tyrphostin(s), which are utilized for eliminating those lymphocytes that respond to the administered antigens.

As is discussed hereinabove and is further exemplified in the Examples section that follows, various tyrphostins of different families can be used in the tolerogenic treatment of the present invention.

In this respect, the present invention further provides a method of determining an activity of a tyrphostin in selective elimination of lymphocytes of a first mammal that are responding to antigens of a second, non-syngeneic, mammal. The method is effected by stimulating hematopoietic cells of a first mammal with first antigens of a second mammal in a presence of and without a tyrphostin. The hematopoietic cells of the first mammal are then exposed to second antigens of the second mammal, without the tyrphostin, and the response of the blood mononuclear cells (BMC) of the first mammal to the antigens of the second mammal is measured. The response of the BMC typically involves cell proliferation can therefore be measured by the uptake of a radioactive nucleotide. To this end, the method can further involve irradiation of the BMC, which enables measuring this uptake in order to determine the above response.

This method of the present invention can be performed both in vitro or in vivo. When performed in vitro, the stimulated hematopoietic cells are preferably isolated mononuclear cells or bone marrow cells of one mammal, while the first and seconds antigens are isolated mononuclear cells or bone marrow cells of another mammal. When performed in vivo, the stimulated hematopoietic cells are mononuclear cells or bone marrow cells and the first and second antigens can be isolated mononuclear cells or bone marrow cells of another mammal. Following the administration of the tyrphostin, the exposure of the cells to the second antigens can be performed in vivo, by subsequent administration of the second antigens to the first mammal. Alternatively, this exposure can be performed in vitro, upon isolation of the cells.

The method of determining an activity of a tyrphostin in the context of the tolerogenic treatment of the present invention can be utilized for screening for the most active tyrphostin to be utilized in a particular treatment.

Based on a very similar approach, the present invention further provides a method of determining an optimal concentration of a tyrphostin for selective elimination of lymphocytes of a first mammal, that are responding to antigens of a second, non-syngeneic, mammal. This method is effected by stimulating hematopoietic cells of a first mammal, as is described hereinabove, in the presence of different concentrations of a tyrphostin. The response measured upon exposing the cells to the second antigens enables determination of the tyrphostin concentration that induce the maximal elimination of lymphocytes.

As is further discussed hereinabove and is also demonstrated in the Examples section that follows, the use of tyrphostins in the tolerogenic treatments of the present invention results in (i) selective elimination of undesirable lymphocytes; and (ii) enhancing immunoresponse to third party antigens and to mitogens.

Without being bound to any theory, it is believed that the overall enhancement of the immune system, which results in enhanced immunoresponse of the residual lymphocytes, following the tolerogenic treatment with tyrphostins, is attributed to the inactivation of a down-regulatory signal transduction pathway by the tyrphostins.

This combined action of tyrphostins, namely enhancing the function of residual lymphocytes following elimination of antigen-reactive lymphocytes by treatment, have never been observed hitherto. Whereas many effective treatments exist for suppression of the immune system, no effective treatment for enhancement of the immune system have been disclosed yet.

The enhancement of the immune system by tyrphostins is highly beneficial in various aspects, such as facilitating the immune reconstitution of patients undergoing bone marrow or organ transplantation, which normally remain with suppressed immune system for several years or for life, and hence are susceptible to infections and secondary malignancy; providing treatment for patients with congenital or acquired immune deficiency as well as in cancer patients with a suppressed immune system as a result of concomitant treatment with cytotoxic agents; providing treatment of patients with persistent viral infections (for example carriers of hepatitis B, hepatitis C, EBV, CMV, HIV-1, and more), bacterial infections (for example tuberculosis) or parasites (for example malaria); and raising resistance in cancer patients against the primary cancer and reduce the incidence of second malignancy. Furthermore, the tyrphostin treatment may be used concomitantly with vaccines to raise the efficacy of the immune response to a given antigen in patients with cancer or infections.

As the tolerogenic treatments of the present invention efficiently utilize tyrphostins as an active ingredient for inducing an immune tolerance, the present invention further provides a pharmaceutical composition that comprises at least one tyrphostin. In particular, the present invention provides a packaged pharmaceutical composition (kit), which comprises, as an active ingredient, an effective amount of one or more tyrphostin(s) and a pharmaceutically acceptable carrier. The pharmaceutical composition is packaged in a package and is identified in print associated with the package for use in an immune tolerance application. The immune tolerance application can be any of the tolerogenic methods described hereinabove.

As used herein a “pharmaceutical composition” refers to a preparation or a composition of one or more of the tyrphostins described herein, or physiologically acceptable salts or prodrugs thereof, with other chemical components such as physiologically suitable carriers and excipients. The purpose of a pharmaceutical composition is to facilitate administration of a compound to an organism.

Hereinafter, the terms “physiologically acceptable carrier” and “pharmaceutically acceptable carrier” which may be interchangeably used refer to a carrier or a diluent that does not cause significant irritation to an organism and does not abrogate the biological activity and properties of the administered compound.

Herein the term “excipient” refers to an inert substance added to a pharmaceutical composition to further facilitate administration of a compound. Examples, without limitation, of excipients include calcium carbonate, calcium phosphate, various sugars and types of starch, cellulose derivatives, gelatine, vegetable oils and polyethylene glycols.

Techniques for formulation and administration of drugs may be found in “Remington's Pharmaceutical Sciences,” Mack Publishing Co., Easton, Pa., latest edition, which is incorporated herein by reference.

Suitable routes of administration of the pharmaceutical composition of the present invention may, for example, include oral, rectal, transmucosal, intestinal or parenteral delivery, including intramuscular, subcutaneous and intramedullary injections as well as intrathecal, direct intraventricular, intravenous, intraperitoneal, intranasal, or intraocular injections.

Pharmaceutical compositions of the present invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with the present invention thus may be formulated in conventional manner using one or more physiologically acceptable carriers comprising excipients and auxiliaries, which facilitate processing of the active compounds into preparations which, can be used pharmaceutically. Proper formulation is dependent upon the route of administration chosen.

For injection, the compounds of the invention may be formulated in aqueous solutions, preferably in physiologically compatible buffers such as Hank's solution, Ringer's solution, or physiological saline buffer.

For transmucosal administration, penetrants appropriate to the barrier to be permeated are used in the formulation. Such penetrants are generally known in the art.

For oral administration, the tyrphostins can be formulated readily by combining same with pharmaceutically acceptable carriers well known in the art. Such carriers enable the active ingredients to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatine, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions, which can be used orally, include push-fit capsules made of gelatine as well as soft, sealed capsules made of gelatine and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by inhalation, the compounds for use according to the present invention are conveniently delivered in the form of an aerosol spray presentation from a pressurized pack or a nebulizer with the use of a suitable propellant, e.g., dichlorodifluoromethane, trichlorofluoromethane, dichlorotetrafluoroethane or carbon dioxide. In the case of a pressurized aerosol, the dosage unit may be determined by providing a valve to deliver a metered amount. Capsules and cartridges of, e.g., gelatin for use in an inhaler or insufflator may be formulated containing a powder mix of the compound and a suitable powder base such as lactose or starch.

The compositions described herein may be formulated for parenteral administration, e.g., by bolus injection or continues infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active tyrphostin in water-soluble form. Additionally, suspensions of the active compounds may be prepared as appropriate oily injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the compounds to allow for the preparation of highly concentrated solutions.

Alternatively, the active ingredient may be in powder form for constitution with a suitable vehicle, e.g., sterile, pyrogen-free water, before use.

The composition of the present invention may also be formulated in rectal compositions such as suppositories or retention enemas, using, e.g., conventional suppository bases such as cocoa butter or other glycerides.

The pharmaceutical compositions herein described may also comprise suitable solid of gel phase carriers or excipients. Examples of such carriers or excipients include, but are not limited to, calcium carbonate, calcium phosphate, various sugars, starches, cellulose derivatives, gelatin and polymers such as polyethylene glycols.

Many of the tyrphostins in the composition of the present invention may be provided as physiologically acceptable salts wherein the tyrphostin compound may form the negatively or the positively charged species. Examples of salts in which the compound forms the positively charged moiety include, without limitation, quaternary ammonium, salts such as the hydrochloride, sulfate, carbonate, lactate, tartrate, maleate, succinate, etc., wherein the nitrogen of the quaternary ammonium group is a nitrogen of a compound of the present invention which reacts with an appropriate acid. Salts in which the compound forms the negatively charged species include, without limitation, the sodium, potassium, calcium and magnesium salts formed by the reaction of a carboxylic acid group in the molecule with the appropriate base (e.g., sodium hydroxide (NaOH), potassium hydroxide (KOH), calcium hydroxide (Ca(OH)₂), etc.).

Pharmaceutical compositions suitable for use in context of the present invention include compositions wherein the active ingredients are contained in an amount effective to achieve the intended purpose. More specifically, a therapeutically effective amount means an amount of tyrphostin(s) effective to prevent, alleviate or ameliorate symptoms of a disease or condition or prolong the survival of the subject being treated.

Determination of a therapeutically effective amount is well within the capability of those skilled in the art, especially in light of the detailed disclosure provided herein.

The therapeutically effective amount or dose of the tyrphostins can be estimated initially from cell culture assays. For example, a dose can be formulated in animal models to achieve a circulating concentration range that includes the IC₅₀ as determined in cell culture (i.e., the concentration of the test compound, which achieves a half-maximal inhibition of the responding lymphocytes). Such information can be used to more accurately determine useful doses in humans.

Toxicity and therapeutic efficacy of the tyrphostins used in context of the present invention can be determined by standard pharmaceutical procedures in cell cultures or experimental animals, e.g., by determining the IC₅₀ and the LD₅₀ (lethal dose causing death in 50% of the tested animals) for a subject compound. The data obtained from these cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p.1).

Dosage amount and interval may be adjusted individually to provide plasma levels of the active moiety which are sufficient to maintain the kinase modulating effects, termed the minimal effective concentration (MEC). The MEC will vary for each preparation, but can be estimated from in vitro data; e.g., the concentration necessary to achieve 50-90% inhibition responding lymphocytes may be ascertained using the assays described herein. Dosages necessary to achieve the MEC will depend on individual characteristics and route of administration. HPLC assays or bioassays can be used to determine plasma concentrations.

Dosage intervals can also be determined using the MEC value. Preparations should be administered using a regimen, which maintains plasma levels above the MEC for 10-90% of the time, preferable between 30-90% and most preferably 50-90%.

The amount of a composition to be administered will, of course, be dependent on the subject being treated, the severity of the anticipated affliction, the manner of administration, the judgement of the prescribing physician, etc.

The pharmaceutical compositions of the present invention is presented in a pack or dispenser device, such as an FDA approved kit, which may contain one or more unit dosage forms containing the active ingredient. The package may, for example, comprise metal or plastic foil, such as a blister package. The package or dispenser device may be accompanied by instructions for administration. The package or dispenser may also be accompanied by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert.

Additional objects, advantages, and novel features of the present invention will become apparent to one ordinarily skilled in the art upon examination of the following examples, which are not intended to be limiting.

Additionally, each of the various embodiments and aspects of the present invention as delineated hereinabove and as claimed in the claims section below finds experimental support in the following examples.

### EXAMPLES

Reference is now made to the following examples, which together with the above descriptions, illustrate the invention in a non limiting fashion.

### Materials and Experimental Methods

Cell sources: Human peripheral blood mononuclear cells (PBMC) were obtained from healthy human donors after separation on Ficoll-Paque density gradient essentially as described in Boyum, A. Scand. J Lab. Invest. 21 (suppl. 97):77 1968. Reagents:

Tyrphostins: Various tyrphostin compounds were synthesized according to known procedures disclosed in U.S. Pat. Nos. 5,196,446, 5,217,999, 5,302,606, 5,656,655, 5,700,822, 5,700,823, 5,712,395, 5,763,441, 5,773,746, 5,789,427, 5,792,771, 5,849,742, 5,932,580, 5,981,569, 5,990,141, 6,126,917, 6,331555, 6,358,951, 6,258,954 and 5,661,147, and in WO 01/34607, WO 99/07701, WO 99/53924, WO 96/29331, WO 92/20642, WO 91/16892, WO 91/16305 and WO 91/16051, which are all incorporated by reference as if fully set forth herein. Table 1 below presents the chemical nomenclature or the tyrphostin nomenclature (namely, the AG number) of some of the tyrphostin compounds and the molecular weights of all the tyrphostin compounds used herein. FIG. 1(a-i) presents the chemical formulas of all of the tyrphostin compounds used herein.

TABLE 1

| Tyrphostin | Formula | MW |
|---|---|---|
| Tyr 1 | N-benzyl-2-cyano-3-(3,4-dihydroxy phenyl)- | 294 |
| | acrylamide | |
| Tyr 2 | N-benzyl-2-cyano-3-[3,4-dihydroxy-5- | 458 |
| | (3-phenyl propyl sulfanyl methyl)-phenyl]- | |
| | acrylamide | |
| Tyr 3 | 4-amino-1-t-butyl-3-(p-toluyl)pyrazolo[3,4- | 281 |
| | d]pyrimidine | |
| Tyr 4 | 4-amino-1-t-butyl-3-(p-chlorophenyl)pyrazolo[3,4- | 301 |
| | d]pyrimidine | |
| Tyr 5 | 2-cyano-3-(3,4-dihydroxy phenyl)-N- | 336 |
| | (4-phenylbutyl)-acrylamide | |
| Tyr 6 | 2-cyano-3-(3,4-dihydroxy-5-phenethylsulfanyl | 486 |
| | methyl-phenyl)-N-(4-phenylbutyl)-acrylamide | |
| Tyr 7 | 2-(3-hydroxy-4-nitro-benzylidene)malononitrile | 215 |
| Tyr 8 | 4-(6,7-dimethoxyquinazoline-4-yl-amino)-benzamide | 361 |
| Tyr 9 | 4-(6,7-dimethoxyquinazoline-4-yl-amino)-phenol | 335 |
| Tyr 10 | AG 2336 | 317 |
| Tyr 11 | AG 2343 | 238 |
| Tyr 12 | AG 1721 | 378 |
| Tyr 13 | AG 2262 | 319 |
| Tyr 14 | AG 2265 | 319 |
| Tyr 15 | 4-amino-1-t-butyl-3-(2-thiophene)pyrazolo[3,4- | 273 |
| | d]pyrimidine | |
| Tyr 16 | 3-(3,5-dimethyl-H-pyrrol-2-yl-methylene)-2-oxo-2,3- | 343 |
| | dihydro-1H-indole-5-sulfonic acid dimethylamide | |
| Tyr 17 | AG 2164 | 393 |
| Tyr 18 | AG 2165 | 311 |
| Tyr 19 | 4-amino-1-t-butyl-3-(3,4-dimethoxyphenyl)- | 327 |
| | pyrazolo[3,4-d]pyrimidine | |
| Tyr 20 | AG 1786 | 280 |
| Tyr 21 | AG 2497 | 284 |
| Tyr 22 | AG 2498 | 308 |
| Tyr 23 | 2-cyano-3-(3,5-di-t-butyl-4-hydroxy phenyl)- | 316 |
| | thioacrylamide | |
| Tyr 24 | 3-amino-5-[1-cyano-2-(1H-indol-3-yl)vinyl]- | 274 |
| | 1H-pyrazole-4-carbonitrile | |
| Tyr 25 | | 305 |
| Tyr 26 | N-benzyl-2-cyano-3-(3,4-dihydroxy phenyl)- | 274 |
| | thioacrylamide | |
| Tyr 27 | | 315 |
| Tyr 28 | 2,3-dicyano-6-phenyl-pyridazine | 206 |
| Tyr 29 | | 273 |
| Tyr 30 | | 397 |
| Tyr 31 | | 234 |
| Tyr 32 | 2-cyano-3-(3,4-dihydroxy-5-iodo phenyl)-N-(4- | 448 |
| | phenyl propyl)-acrylamide | |
| Tyr 33 | 2-(4-methoxy-benzylidene)malononitrile | 184 |
| Tyr 34 | 2-cyano-3-(3,4-dihydroxyphenyl)-thioacrylamide | 220 |
| Tyr 35 | | 337 |
| Tyr 36 | | 363 |
| Tyr 37 | | 335 |

The tyrphostins were dissolved in dimethylsulfoxide (DMSO) and were diluted in culture medium. The final concentration of each tyrphostin used is indicated in each experiment.

Mitogens: Phytohemagglutinin (PHA) and concanavalin A (Con-A) were dissolved in saline and diluted to their final concentration in culture medium. Anti-CD3 (OK3 stock solution 1 μg/ml) was diluted to 0.2 μg/ml final concentration in culture medium.

Culture conditions: RPMI 1640 medium (Beit Haemek, Israel) was supplemented with 100 U/ml penicillin, 100 μg/ml streptomycin and 2 mM L-glutamine. All cultures were incubated in a humidified incubator at 37° C. in 5% CO₂.

Primary Response of Human PBMC:

One-way mixed lymphocyte reaction (MLR assay): Responding cells derived from donor A (5×10⁴) were cultured in round-bottom microwell plates with an equal number (5×10⁴) of irradiated (6000 cGy) donor B PBMC with and without tyrphostins. Cultures were incubated for six days in a total volume of 0.2 ml culture medium supplemented with 15% heat-inactivated human AB⁺ serum, glutamin 2 mM, and antibiotics (Penicillin 100 Units/ml, Streptomycin 100 μg/ml and Gentamycin 50 μg/ml) in 5% CO₂ in air humidified incubator at 37° C. (the increased serum concentration achieves a better MLR response). During the last 18 hours of incubation, cultures were pulsed with 2 μCi of [³H] TdR.

Primary Mitogenic Response Assay:

Responding PBMC cells (10⁵) derived from donor A were cultured in flat-bottom microwell plates with and without tyrphostins, and with phytohemagglutinin (PHA) 1 μg/ml, or concanavalin A 18 μg/ml. Cultures were incubated for 4 days in a total volume of 0.2 ml RPMI 1640 culture medium supplemented with 10% heat-inactivated human AB⁺ serum, glutamin and antibiotics in a 5% CO₂ in air humidified incubator at 37° C. During the last 18 hours of incubation, cultures were pulsed with 1 μCi of [³H] TdR.

Secondary MLR and Mitogenic Responses of Human PBMC:

PBMC from donor A (10×10⁶) were stimulated with equal number of irradiated (6000 cGy) donor B PBMC, with and without tyrphostins, in a total volume of 20 ml culture medium supplemented with 10% heat inactivated human AB⁺ serum (mixed lymphocyte culture, MLC). Cultures were placed vertically in 25 cm tissue culture flasks in a 5% CO₂, humidified incubator at 37° C. After 10 days, cells were washed twice to remove the tyrphostin, and tested for their ability to respond in the MLR and the primary mitogenic response assay described hereinabove.

The alloreactivity of donor A anti-donor B-primed cells (5×10⁴) was tested in a 6-day one-way primary MLR assay against third party unrelated donor C PBMC (10⁵), or against donor A cells (background control). Cultures were placed in round-bottom microwell plates. On day 5, cultures were pulsed with 2 μCi [³H] TdR/well for 16-18 hours and then harvested. Secondary MLR against donor B PBMC was assayed by incubating 2×10⁴ donor A anti-donor B-primed cells with 10⁵ irradiated donor A or donor B cells in round-bottom microwell plates in a total volume of 0.2 ml culture medium supplemented with 15% heat-inactivated AB⁺ serum. After 48 hours at 37° C. in a 5% CO₂ humidified incubator, cultures were pulsed with 2 μCi [³H] TdR for another 16-18 hours and then harvested.

The mitogenic responses of donor A anti-donor B-primed cells were assayed as follows: Allosensitized cells (105) were cultured in flat-bottom microwell plates with 1-3 μg/ml PHA (depending on batch) or 18 μg/ml Con A or 0.2 μg/ml anti-CD3 antibody. Cultures were incubated for 48 hours in a total volume of 0.2 ml culture medium supplemented with 10% heat-inactivated human AB⁺ serum at 37° C. in a 5% CO₂ humidified incubator, pulsed for 16-18 hours with 1 μCi [³H]TdR/well, and then harvested.

Harvesting and radioactivity determination: Harvesting was carried out by a multiple-sample cell harvester and radioactivity, expressed in counts per minutes (cpm), was determined in a liquid scintillation β-counter.

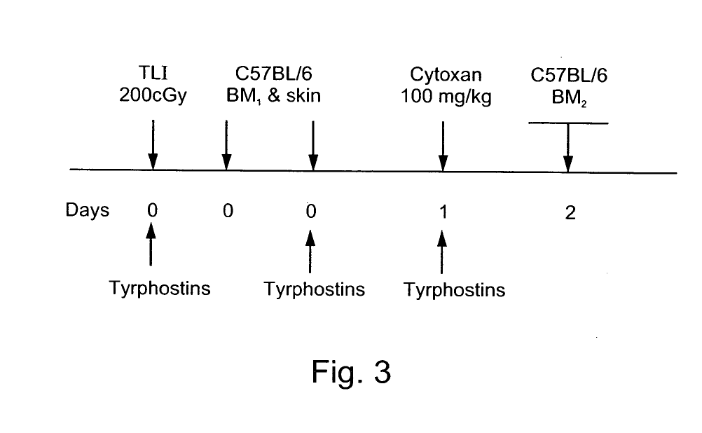

In vivo studies: A schematic description of the in vivo procedure is presented in FIG. 3. Mice were conditioned prior to the tolerogenic treatment by a single TLI fraction of 200 cGy, using known procedures [Slavin et al., J. Exp. Med., 146:34 (1997); Prigozhina et al. Transplantation 63:(10):1394 (1997); Prigozhina et al. Experimental Hematology 27:1503 (1999); Prigozhina et al. Exp. Hematol. 30(1) (2002)]. On the same day, the mice were administered, subcutaneously, with the first injection of tyrphostin or tyrphostins, with a maximal dose of 50 μl in DMSO. Following an inoculation of 3×10⁷ donor bone marrow cells and donor skin transplantation, a second tyrphostin injection was given on the same day, at the end of the transplant procedure. A third injection of tyrphostin was given on day +1, and subsequently mice were injected with a sub-optimal dose of cyclophosphamide (100 mg/kg, which by itself was shown to be ineffective). On day +2, all mice were administered with a second infusion of 30×10⁶ donor bone marrow, intravenously.

Skin grafting was carried out on day 0, as described hereinabove (tolerance was tested as preclinical model for cadaveric organ transplantation when the donor becomes available with no prior notice). A full-thickness skin graft measuring 1 cm×1 cm was adjusted to the graft bed by 4 Thomas surgery clips (Thomas Scientific, USA). The panniculus carnosus was kept intact in the graft bed. The graft was considered to be accepted when hair of donor color grew on the soft flexible underlying skin, and rejected when donor epithelium was lost.

Inhibition of Primary MLR by Tyrphostins:

Human PBMC were incubated either with autologous or with MHC-mismatched PBMC in a one-way MLR assay, with and without various concentrations of tyrphostins, as is described hereinabove.

Tables 2-4 below present the effect of various concentrations of a variety of tyrphostins on primary MLR.

TABLE 2

| | Concentration | | % |
|---|---|---|---|
| Tyrphostin | (μM) | cpm | Inhibition |
| None | — | 10,734 | 0 |
| Tyr 1 | 25 | 2,420 | 77 |
| | 50 | 1,132 | 89 |
| Tyr 2 | 20 | 2,863 | 73 |
| | 40 | 490 | 95 |
| Tyr 3 | 5 | 3,130 | 61 |
| | 10 | 145 | 99 |
| Tyr 4 | 5 | 3,316 | 69 |
| | 10 | 580 | 95 |
| Tyr 5 | 10 | 6,413 | 40 |
| | 50 | 3,156 | 70 |
| Tyr 6 | 10 | 4,431 | 59 |
| | 50 | 168 | 99 |
| Tyr 7 | 50 | 5,348 | 50 |
| | 100 | 512 | 95 |

TABLE 3

| | | | |
|---|---|---|---|
| | Concentration | | % |
| Tyrphostin | (μM) | cpm ± SD | Inhibition |

| None | — | 30,520 ± | 2417 | 0 |
|---|---|---|---|---|
| Tyr 8 | 25 | 12,452 ± | 5460 | 49 |
| | 50 | 11,293 ± | 2268 | 63 |
| Tyr 9 | 25 | 1,367 ± | 183 | 95 |
| | 50 | 371 ± | 9 | 99 |
| Tyr 10 | 1 | 3,968 ± | 1335 | 87 |
| | 25 | 1,658 ± | 390 | 95 |
| Tyr 11 | 25 | 8,811 | | 70 |
| | 50 | 3,691 ± | 1613 | 78 |

TABLE 4

| | Concentration | % |
|---|---|---|
| Tyrphostin | (μM) | Inhibition |
| None | — | 0 |
| Tyr 12 | 25 | 70 |
| | 50 | 92 |
| Tyr 16 | 1 | 62 |
| Tyr 18 | 20 | 80 |
| | 40 | 90 |
| Tyr 19 | 10 | 63 |
| | 20 | 80 |

Table 5 below presents the IC50 values obtained for inhibition of Primary MLR by Tyr 1-Tyr 20.

TABLE 5

| | IC 50% | |
|---|---|---|
| Tyrphostin | (μM) | |
| Tyr 1 | 45 | (mean) |
| Tyr 2 | 22 | (mean) |
| Tyr 3 | 5 | (mean) |
| Tyr 4 | 5 | (mean) |
| Tyr 5 | 34 | (mean) |
| Tyr 6 | 23 | (mean) |
| Tyr 7 | 36 | (mean) |
| Tyr 8 | 43 | |
| Tyr 9 | 26 | |
| Tyr 10 | 0.8 | |
| Tyr 11 | 30 | |
| Tyr 12 | 23 | |
| Tyr 13 | 56 | |
| Tyr 14 | 81 | |
| Tyr 15 | 7 | |
| Tyr 16 | 4 | |
| Tyr 17 | 15 | (mean) |
| Tyr 18 | 8 | (mean) |
| Tyr 19 | 3 | |
| Tyr 20 | 22 | |

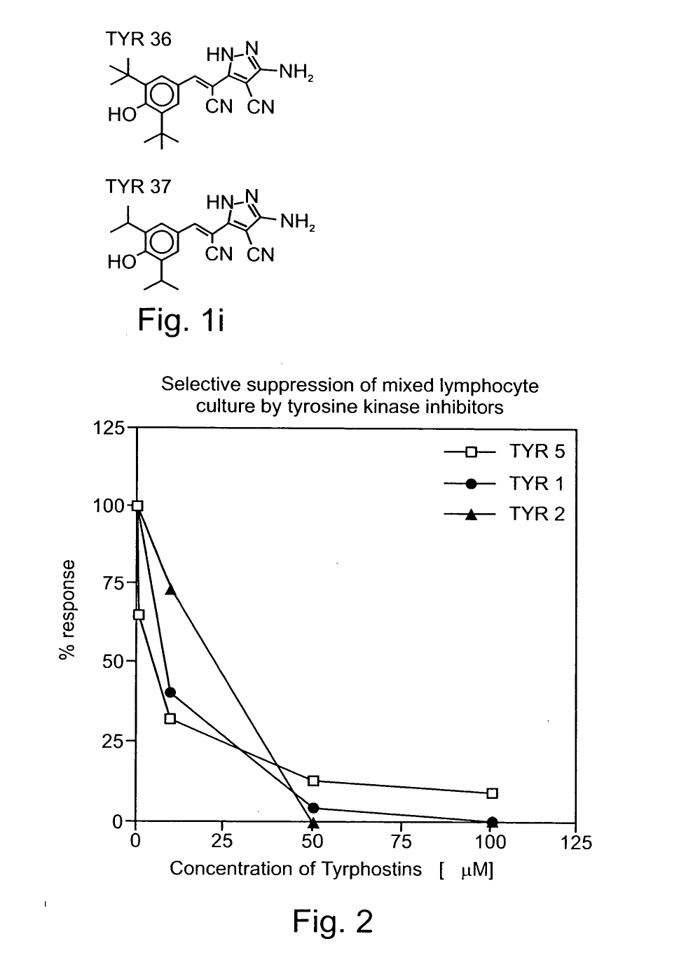

FIG. 2 further demonstrates the inhibition of primary MLR by three tyrphostins—Tyr 1, Tyr 2 and Tyr 5, by presenting the alloreactive relative response that was observed with each of the tyrphostins. In this case, the alloreactive response without the tyrphostins was considered as 100% response. Percentage of response was calculated according to the following formula: b/a×100; where a=cpm in cultures without tyrphostin, b=cpm in cultures with tyrphostin.

As is clearly shown in FIG. 2, inhibition of alloreactivity was already observed in the presence of 10 μM of Tyr 1 and Tyr 5 (40% and 32% response, respectively). Higher concentrations, e.g., 50 μM, almost totally eliminated the response (4% and 13%, respectively). A low concentration of Tyr 2 (10 μM) was not very effective in inhibiting primary MLR (73% response) but 50 μM Tyr 2 already achieved a total inhibition of alloreactivity (0% response). Controls tests of appropriate DMSO concentrations which correlate to 50 μM tyrphostin did not affect proliferation response in MLR or mitogenic assays.

These results show that the response of PBMC to MHC-mismatched cells could be effectively abrogated in the presence of a variety of tyrphostins.

Inhibition of Mitogenic Response by Tyrphostins:

Table 6 below presents the inhibition effect of various tyrphostins on mitogenic responses, following the primary mitogenic response assay described hereinabove.

TABLE 6

| Tyrphostin | | % |
|---|---|---|
| (20 μM) | cpm ± SD | Inhibition |
| Tyr 1 | 6,023 ± 876 | 60 |
| Tyr 3 | 300 ± 24 | 98 |
| Tyr 4 | 230 ± 115 | 98 |
| Tyr 6 | 4,662 ± 104 | 70 |
| Tyr 9 | 4,582 ± 617 | 70 |
| Tyr 19 | 553 ± 59 | 96 |
| Tyr 23 | 4,831 ± 1026 | 68 |
| Tyr 24 | 533 ± 45 | 96 |
| Tyr 26 | 4,295 ± 221 | 70 |
| Tyr 28 | 5,159 ± 240 | 66 |
| Tyr 32 | 6,303 ± 1205 | 58 |

Without tyrphostin: medium 155±37

PHA 14,959±644

Alloreactivity and Mitogenic Responses of Alloantigen-Primed PBMC (Secondary MLR):

PBMC of donor A were primed in MLC with PBMC of donor B in the presence of 20-50 μM tyrphostin. After 10 days the tyrphostin was removed and the primed cells were tested for their ability to respond in a secondary MLR assay against the priming (donor B) alloantigens, in a primary MLR assay against third party unrelated alloantigens (donor C), and to non-specific stimulation in a mitogenic assay. Priming without tyrphostins resulted in a proliferative response which was considered as 100% response. Percentage of response was calculated according to the formula described above.

Table 7 below presents the effect of various tyrphostins of the alloreactivity and mitogenic response of allosensitized human PBMC (primed cells).

TABLE 7

| | | % Response to | % Response | |
|---|---|---|---|---|
| | Concentration | primary | to unrelated | % Response |
| Tyrphostin | (μM) | stimulator | stimulator | to PHA |
| Tyr 1 | 25 | 22 ± 14 | 99 ± 29 | 290 ± 111 |
| | 50 | 19 ± 10 | 175 ± 77 | 176 ± 50 |
| Tyr 3 | 5 | 65 | 55 | 26 |
| | 10 | 24 ± 10 | 206 ± 99 | 110 ± 35 |
| | 20 | 24 ± 18 | 57 ± 44 | 30 ± 28 |
| Tyr 4 | 5 | 132 | 145 | 29 |
| | 10 | 13 ± 4.5 | 288 ± 122 | 120 ± 40 |
| | 20 | 6.5 ± 0.5 | 204 ± 18 | 40 ± 32 |
| Tyr 2 | 20 | 280 | 257 | 900 |
| | 40 | 15 | 138 | 254 |
| Tyr 6 | 10 | 32 | 250 | 152 |
| | 20 | 32 ± 0 | 220 ± 93 | 62 ± 42 |
| | 30 | 46 | 185 | 20 |

The results obtained clearly demonstrate that in the presence of tryphostins, the ability of the primed cells to react against the priming donor cells was significantly reduced, while their response to third party unrelated donor cells was generally enhanced. The results further demonstrate that the allosensitized cells that were primed in the presence of tyrphostins retained their ability to be stimulated by T-cell mitogens such as PHA.

Hence, the above results show that the presence of tyrphostins during the allosensitization phase selectively inhibits the ability of the primed cells to react against the sensitizing alloantigens. The selective inhibition spares other non-activated T cells which can subsequently mount an alloreactive response against third party unrelated alloantigens or react to non-specific mitogenic stimuli. This selective mode of action of the tyrphostins can be exploited to achieve clonal-specific inactivation of alloreactivity without impairing the functions of other T cell subsets.

Skin Grafting:

Table 8 below presents the results obtained in the skin grafting procedure described hereinabove and schematically presented in FIG. 3. The results present survival.

TABLE 8

| | | | Number | | | | |
|---|---|---|---|---|---|---|---|
| | Quantity | | of | Survivors | | Survivors | |
| | per | | injec- | after | | after | |
| Tyrphostin(s) | injection | | tions | 50 days | | 100 days | |
| None | | | | 3/17 | (18%) | 2/16 | (13%) |
| Tyr 1 | 400 | mg | | 1/5 | (20%) | 1/5 | (20%) |
| | in 15 | μl | | | | | |
| Tyr 33 | 300 | mg | | 2/6 | (30%) | 2/6 | (30%) |
| | in 30 | μl | | | | | |
| Tyr 1 | 200 | μg | 3 | 10/12 | (83%) | 10/12 | (83%) |
| Tyr 34 | 200 | μg | 3 | | | | |
| Tyr 1 | 200 | μg | 3 | | | | |
| Tyr 7 | 500 | μg | 3 | 5/5 | (100%) | 5/5 | (100%) |
| Tyr 33 | 300 | μg | 3 | | | | |
| Tyr 34 | 200 | μg | 3 | | | | |

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

What is claimed is:

1. A method of inducing immune tolerance in a first mammal to antigens of a second, non-syngeneic, mammal, the method comprising:
administering antigens from said second mammal to said first mammal; and
administering a non-myeloablative dose of at least one tyrphostin to said first mammal to selectively eliminate mammal lymphocytes responding to said antigens.

2. The method of claim 1, further comprising, prior to, or concomitant with, administering said antigens from said second mammal:
administering at least one immunosuppressive agent to said first mammal in a non-myeloablative regimen sufficient to decrease the functional T lymphocyte population of said first mammal.

3. The method of claim 1, wherein said antigens from said second mammal comprise one or more antigens selected from the group consisting of non-cellular antigens, cells, organs and tissues, either alive or killed.

4. The method of claim 3, wherein said antigens from said second mammal comprise hematopoietic cells.

5. The method of claim 2, wherein said at least one immunosuppressive agent is selected from the group consisting of an immunosuppressive drug, an alkylating agent, ionizing radiation and anti-leukocyte or anti-leukocyte function antibodies.

6. The method of claim 5, wherein said at least one immunosuppressive agent is a short-course total lymphoid irradiation (sTLI).

7. The method of claim 6, wherein said sTLI comprises administration of 1-12 doses of 200 cGy.

8. The method of claim 2, wherein said antigens from said second mammal comprise hematopoietic stem cells and said decrease of said T lymphocyte population is to a level permitting at least transient survival of said hematopoietic stem cells.

9. The method of claim 8, wherein said decrease of said T lymphocyte population is at least about 90%.

10. The method of claim 9, wherein said decrease of said T lymphocyte population is at least about 95%.

11. The method of claim 10, wherein said decrease of said T lymphocyte population is at least about 99%.

12. The method of claim 1, wherein said second, non-syngeneic, mammal is an allogeneic mammal.

13. The method of claim 1, wherein said second, non-syngeneic, mammal is a xenogeneic mammal.

14. The method of claim 1, wherein said at least one tyrphostin is of a family selected from the group consisting of quinoxalines, quinazolines, cyano-substituted acrylamides, cyano-substituted thioacrylamides, acrylonitriles, phenyl-substituted acrylonitriles, substituted anilines, benzoxazolones, tricyclic pyridones and tetracyclic pyridones.

15. The method of claim 1, wherein said at least one tyrphostin is selected from the group consisting of: N-benzyl-2-cyano-3-(3,4-dihydroxyphenyl)-acrylamide, N-benzyl-2-cyano-3-[3,4-dihydroxy-5-(3-phenylpropylsulfanylmethyl)-phenyl]-acrylamide, 4-amino-1-t-butyl-3-(p-toluyl)pyrazolo[3,4-d]pyrimidine, 4-amino-1-t-butyl-3-(p-chlorophenyl)pyrazolo[3,4-d]pyrimidine, 2-cyano-3-(3,4-dihydroxyphenyl)-N-(4-phenylbutyl)-acrylamide, 2-cyano-3-(3,4-dihydroxy-5-phenethylsulfanylmethyl-phenyl)-N-(4-phenylbutyl)-acrylamide, 2-(3-hydroxy-4-nitro-benzylidene)malononitrile, 4-(6,7-dimethoxyquinazoline-4-yl-amino)-benzamide, 4-(6,7-dimethoxyquinazoline-4-yl-amino)-phenol, 4-amino-1-t-butyl-3-(2-thiophene)pyrazolo[3,4-d]pyrimidine, 3-(3,5-dimethyl-H-pyrrol-2-yl-methylene)-2-oxo-2,3-dihydro-1H-indole-5-sulfonic acid dimethylamide, 4-amino-1-t-butyl-3-(3,4-dimethoxyphenyl)pyrazolo[3,4-d]pyrimidine, 2-cyano-3-(3,5-di-t-butyl-4-hydroxy phenyl)-thioacrylamide, 3-amino-S-[1-cyano-2-(1H-indol-3-yl)vinyl]-1H-pyrazole-4-carbonitrile, N-benzyl-2-cyano-3-(3,4-dihydroxy phenyl)-thioacrylamide, 2,3-dicyano-6-phenyl-pyridazine, 2-cyano-3-(3,4-dihydroxy-5-iodophenyl)-N-(4-phenyl propyl)-acrylamide, 2-(4-methoxy-benzylidene)malononitrile, and 2-cyano-3-(3,4-dihydroxyphenyl)-thioacrylamide.

16. A method of inducing immune tolerance in a first mammal to antigens of a second, non-syngeneic, mammal, and transplanting a graft derived from the second mammal in the first mammal, while minimizing graft rejection, the method comprising:
administering antigens from said second mammal to said first mammal;
administering a non-myeloablative dose of at least one tyrphostin to said first mammal to selectively eliminate mammal lymphocytes responding to said antigens; and
transplanting the graft in the first mammal.

17. The method of claim 16, further comprising, prior to, or concomitant with, administering said antigens from said second mammal:
administering at least one immunosuppressive agent to said first mammal in a non-myeloablative regimen sufficient to decrease the functional T lymphocyte population of said first mammal.

18. The method of claim 16, wherein said antigens from said second mammal comprise one or more antigens selected from the group consisting of non-cellular antigens, cells, organs and tissues, either alive or killed.

19. The method of claim 18, wherein said antigens from said second mammal comprise hematopoietic cells.

20. The method of claim 17, wherein said at least one immunosuppressive agent is selected from the group consisting of an immunosuppressive drug, an alkylating agent, ionizing radiation and anti-leukocyte or anti-leukocyte function antibodies.

21. The method of claim 20, wherein said at least one immunosuppressive agent is a short-course total lymphoid irradiation (sTLI).

22. The method of claim 21, wherein said sTLI comprises administration of 1-12 doses of 200 cGy.

23. The method of claim 17, wherein said antigens from said second mammal comprise hematopoietic stem cells and said decrease of said T lymphocyte population is to a level permitting at least transient survival of said hematopoietic stem cells.

24. The method of claim 23, wherein said decrease of said T lymphocyte population is at least about 90%.

25. The method of claim 24, wherein said decrease of said T lymphocyte population is at least about 95%.

26. The method of claim 25, wherein said decrease of said T lymphocyte population is at least about 99%.

27. The method of claim 16, wherein said second, non-syngeneic, mammal is an allogeneic mammal.

28. The method of claim 16, wherein said second, non-syngeneic, mammal is a xenogeneic mammal.

29. The method of claim 16, wherein said graft is an organ or a tissue is not rich in immunocompetent lymphocytes.

30. The method of claim 16, further comprising administering a preparation of hematopoietic stem cells from said non-syngeneic second mammal to said first mammal.

31. The method of claim 30, further comprising, following administering said preparation:
treating said first mammal with allogeneic cell therapy, said allogeneic cell therapy comprises infusing allogeneic lymphocytes from said donor into said host mammal.

32. The method of claim 30, wherein the blood of said first mammal comprises at least 20% cells of said second mammal.

33. The method of claim 27, wherein said first mammal is a human patient.

34. The method of claim 28, wherein said first mammal is a human patient.

35. The method of claim 30, wherein said first mammal is a cancer patient.

36. The method of claim 16, wherein said at least one tyrphostin is of a family selected from the group consisting of quinoxalines, quinazolines, cyano-substituted acrylamides, cyano-substituted thioacrylamides, acrylonitriles, phenyl-substituted acrylonitriles, substituted anilines, benzoxazolones, tricyclic pyridones and tetracyclic pyridones.

37. The method of claim 16, wherein said at least one tyrphostin is selected from the group consisting of: N-benzyl-2-cyano-3-(3,4-dihydroxyphenyl)-acrylamide, N-benzyl-2-cyano-3-[3,4-dihydroxy-5-(3-phenylpropylsulfanylmethyl)-phenyl]-acrylamide, 4-amino-1-t-butyl-3-(p-toluyl)pyrazolo[3,4-d]pyrimidine, 4-amino-1-t-butyl-3-(p-chlorophenyl)pyrazolo[3,4-d]pyrimidine, 2-cyano-3-(3,4-dihydroxyphenyl)-N-(4-phenylbutyl)-acrylamide, 2-cyano-3-(3,4-dihydroxy-5-phenethylsulfanylmethyl-phenyl)-N-(4-phenylbutyl)-acrylamide, 2-(3-hydroxy-4-nitro-benzylidene)malononitrile, 4-(6,7-dimethoxyquinazoline-4-yl-amino)-benzamide, 4-(6,7-dimethoxyquinazoline-4-yl-amino)-phenol, 4-amino-1-t-butyl-3-(2-thiophene)pyrazolo[3,4-d]pyrimidine, 3-(3,5-dimethyl-H-pyrrol-2-yl-methylene)-2-oxo-2,3-dihydro-1H-indole-5-sulfonic acid dimethylamide, 4-amino-1-t-butyl-3-(3,4-dimethoxyphenyl)pyrazolo[3,4-d]pyrimidine, 2-cyano-3-(3,5-di-t-butyl-4-hydroxy phenyl)-thioacrylamide, 3-amino-5-[1-cyano-2-(1H-indol-3-yl)vinyl]-1H-pyrazole-4-carbonitrile, N-benzyl-2-cyano-3-(3,4-dihydroxy phenyl)-thioacrylamide, 2,3-dicyano-6-phenyl-pyridazine, 2-cyano-3-(3,4-dihydroxy-5-iodophenyl)-N-(4-phenyl propyl)-acrylamide, 2-(4-methoxy-benzylidene)malononitrile, and 2-cyano-3-(3,4-dihydroxyphenyl)-thioacrylamide.

38. A method of inducing immune tolerance in a first mammal to antigens of a second, non-syngeneic, mammal, and transplanting a graft derived from the first mammal in the second mammal while reducing graft-versus-host disease, the method comprising:
administering antigens from said second mammal to said first mammal;
administering a non-myeloablative dose of at least one tyrphostin to said first mammal to selectively eliminate mammal lymphocytes responding to said antigens; and
transplanting the graft in the second mammal.

39. The method of claim 38, wherein said graft is rich in immunocompetent lymphocytes.

40. The method of claim 39, wherein said graft is selected from the group consisting of bone marrow cells, small intestine and pancreatic islets.

41. The method of claim 38, further comprising, prior to, or concomitant with, administering said antigens from said second mammal:
administering at least one immunosuppressive agent to said first mammal in a non-myeloablative regimen sufficient to decrease the functional T lymphocyte population of said mammal.

42. The method of claim 38, wherein said antigens from said second mammal comprise one or more antigens selected from the group consisting of non-cellular antigens, cells, organs and tissues, either alive or killed.

43. The method of claim 42, wherein said antigens from said second mammal comprise hematopoietic cells.

44. The method of claim 41, wherein said at least one immunosuppressive agent is selected from the group consisting of an immunosuppressive drug, an alkylating agent, ionizing radiation and anti-leukocyte or anti-leukocyte function antibodies.

45. The method of claim 44, wherein said at least one immunosuppressive agent is a short-course total lymphoid irradiation (sTLI).

46. The method of claim 45, wherein said sTLI comprises administration of 1-12 doses of 200 cGy.

47. The method of claim 41, wherein said antigens from said second mammal comprise hematopoietic stem cells and said decrease of said T lymphocyte population is to a level permitting at least transient survival of said hematopoietic stem cells.

48. The method of claim 47, wherein said decrease of said T lymphocyte population is at least about 90%.

49. The method of claim 48, wherein said decrease of said T lymphocyte population is at least about 95%.

50. The method of claim 49, wherein said decrease of said T lymphocyte population is at least about 99%.

51. The method of claim 38, wherein said second, non-syngeneic, mammal is an allogeneic mammal.

52. The method of claim 51, wherein said second, non-syngeneic, mammal is a xenogeneic mammal.

53. The method of claim 51, wherein said first mammal is a human patient.

54. The method of claim 52, wherein said first mammal is a human patient.

55. The method of claim 52, wherein said second mammal is a human patient.

56. The method of claim 53, wherein said first mammal is a cancer patient.

57. The method of claim 54, wherein said first mammal is a cancer patient.

58. The method of claim 38, wherein said at least one tyrphostin is of a family selected from the group consisting of quinoxalines, quinazolines, cyano-substituted acrylamides, cyano-substituted thioacrylamides, acrylonitriles, phenyl-substituted acrylonitriles, substituted anilines, benzoxazolones, tricyclic pyridones and tetracyclic pyridones.

59. The method of claim 38, wherein said at least one tyrphostin is selected from the group consisting of: N-benzyl-2-cyano-3-(3,4-dihydroxyphenyl)-acrylamide, N-benzyl-2-cyano-3-[3,4-dihydroxy-5-(3-phenylpropylsulfanylmethyl)-phenyl]-acrylamide, 4-amino-1-t-butyl-3-(p-toluyl)pyrazolo[3,4-d]pyrimidine, 4-amino-1-t-butyl-3-(p-chlorophenyl)pyrazolo[3,4-d]pyrimidine, 2-cyano-3-(3,4-dihydroxyphenyl)-N-(4-phenylbutyl)-acrylamide, 2-cyano-3-(3,4-dihydroxy-5-phenethylsulfanylmethyl-phenyl)-N-(4-phenylbutyl)-acrylamide, 2-(3-hydroxy-4-nitro-benzylidene)malononitrile, 4-(6,7-dimethoxyquinazoline-4-yl-amino)-benzamide, 4-(6,7-dimethoxyquinazoline-4-yl-amino)-phenol, 4-amino-1-t-butyl-3-(2-thiophene)pyrazolo[3,4-d]pyrimidine, 3-(3,5-dimethyl-H-pyrrol-2-yl-methylene)-2-oxo-2,3-dihydro-1H-indole-5-sulfonic acid dimethylamide, 4-amino-1-t-butyl-3-(3,4-dimethoxyphenyl)pyrazolo[3,4-d]pyrimidine, 2-cyano-3-(3,5-di-t-butyl-4-hydroxy phenyl)-thioacrylamide, 3-amino-5-[1-cyano-2-(1H-indol-3-yl)vinyl]-1H-pyrazole-4-carbonitrile, N-benzyl-2-cyano-3-(3,4-dihydroxy phenyl)-thioacrylamide, 2,3-dicyano-6-phenyl-pyridazine, 2-cyano-3-(3,4-dihydroxy-5-iodophenyl)-N-(4-phenyl propyl)-acrylamide, 2-(4-methoxy-benzylidene)malononitrile, and 2-cyano-3-(3,4-dihydroxyphenyl)-thioacrylamide.

60. A method of inducing bilateral immune tolerance in a first mammal and a second, non-syngeneic, second mammal, the method comprising:
administering antigens from said second mammal to said first mammal;
administering a non-myeloablative dose of at least one tyrphostin to said first mammal to selectively eliminate mammal lymphocytes responding to said antigens, thereby inducing immune tolerance in the first mammal to antigens of the second mammal;
administering antigens from said first mammal to said second mammal; and
administering a non-myeloablative dose of at least one tyrphostin to said second mammal to selectively eliminate mammal lymphocytes responding to said antigens, thereby inducing immune tolerance in the second mammal to antigens of the first mammal.

61. The method of claim 60, further comprising, prior to, or concomitant with, administering said antigens from said second mammal:
administering at least one immunosuppressive agent to said first mammal in a non-myeloablative regimen sufficient to decrease the functional T lymphocyte population of said mammal.

62. The method of claim 60, wherein said antigens from said second mammal comprise one or more antigens selected from the group consisting of non-cellular antigens, cells, organs and tissues, either alive or killed.

63. The method of claim 62, wherein said antigens from said second mammal comprise hematopoietic cells.

64. The method of claim 61, wherein said at least one immunosuppressive agent is selected from the group consisting of an immunosuppressive drug, an alkylating agent, ionizing radiation and anti-leukocyte or anti-leukocyte function antibodies.

65. The method of claim 64, wherein said at least one immunosuppressive agent is a short-course total lymphoid irradiation (sTLI).

66. The method of claim 65, wherein said sTLI comprises administration of 1-12 doses of 200 cGy.

67. The method of claim 61, wherein said antigens from said second mammal comprise hematopoietic stem cells and said decrease of said T lymphocyte population is to a level permitting at least transient survival of said hematopoietic stem cells.

68. The method of claim 67, wherein said decrease of said T lymphocyte population is at least about 90%.

69. The method of claim 68, wherein said decrease of said T lymphocyte population is at least about 95%.

70. The method of claim 69, wherein said decrease of said T lymphocyte population is at least about 99%.

71. The method of claim 60, further comprising, prior to, or concomitant with, administering said antigens from first mammal:
administering at least one immunosuppressive agent to said second mammal in a non-myeloablative regimen sufficient to decrease the functional T lymphocyte population of said mammal.

72. The method of claim 60, wherein said antigens from said first mammal comprise one or more antigens selected from the group consisting of non-cellular antigens, cells, organs and tissues, either alive or killed.

73. The method of claim 62, wherein said antigens from said first mammal comprise hematopoietic stem cells.

74. The method of claim 71, wherein said at least one immunosuppressive agent is selected from the group consisting of an immunosuppressive drug, an alkylating agent, ionizing radiation and anti-leukocyte or anti-leukocyte function antibodies.

75. The method of claim 74, wherein said at least one immunosuppressive agent is a short-course total lymphoid irradiation (sTLI).

76. The method of claim 75, wherein said sTLI comprises administration of 1-12 doses of 200 cGy.

77. The method of claim 71, wherein said antigens from said first mammal comprise hematopoietic stem cells and said decrease of said T lymphocyte population is to a level permitting at least transient survival of said hematopoietic stem cells.

78. The method of claim 77, wherein said decrease of said T lymphocyte population is at least about 90%.

79. The method of claim 78, wherein said decrease of said T lymphocyte population is at least about 95%.

80. The method of claim 79, wherein said decrease of said T lymphocyte population is at least about 99%.

81. The method of claim 60, wherein said second, non-syngeneic, mammal is an allogeneic mammal.

82. The method of claim 60, wherein said second, non-syngeneic, mammal is a xenogeneic mammal.

83. The method of claim 60, wherein said at least one tyrphostin is of a family selected from the group consisting of quinoxalines, quinazolines, cyano-substituted acrylamides, cyano-substituted thioacrylamides, acrylonitriles, phenyl-substituted acrylonitriles, substituted anilines, benzoxazolones, tricyclic pyridones and tetracyclic pyridones.

84. The method of claim 60, wherein said at least one tyrphostin is selected from the group consisting of: N-benzyl-2-cyano-3-(3,4-dihydroxyphenyl)-acrylamide, N-benzyl-2-cyano-3-[3,4-dihydroxy-5-(3-phenylpropylsulfanylmethyl)-phenyl]-acrylamide, 4-amino-1-t-butyl-3-(p-toluyl)pyrazolo[3,4-d]pyrimidine, 4-amino-1-t-butyl-3-(p-chlorophenyl)pyrazolo[3,4-d]pyrimidine, 2-cyano-3-(3,4-dihydroxyphenyl)-N-(4-phenylbutyl)-acrylamide, 2-cyano-3-(3,4-dihydroxy-5-phenethylsulfanylmethyl-phenyl)-N-(4-phenylbutyl)-acrylamide, 2-(3-hydroxy-4-nitro-benzylidene)malononitrile, 4-(6,7-dimethoxyquinazoline-4-yl-amino)-benzamide, 4-(6,7-dimethoxyquinazoline-4-yl-amino)-phenol, 4-amino-1-t-butyl-3-(2-thiophene)pyrazolo[3,4-d]pyrimidine, 3-(3,5-dimethyl-H-pyrrol-2-yl-methylene)-2-oxo-2,3-dihydro-1H-indole-5-sulfonic acid dimethylamide, 4-amino-1-t-butyl-3-(3,4-dimethoxyphenyl)pyrazolo[3,4-d]pyrimidine, 2-cyano-3-(3,5-di-t-butyl-4-hydroxy phenyl)-thioacrylamide, 3-amino-5-[1-cyano-2-(1H-indol-3-yl)vinyl]-1H-pyrazole-4-carbonitrile, N-benzyl-2-cyano-3-(3,4-dihydroxy phenyl)-thioacrylamide, 2,3-dicyano-6-phenyl-pyridazine, 2-cyano-3-(3,4-dihydroxy-5-iodophenyl)-N-(4-phenyl propyl)-acrylamide, 2-(4-methoxy-benzylidene)malononitrile, and 2-cyano-3-(3,4-dihydroxyphenyl)-thioacrylamide.

85. A method of inducing bilateral immune tolerance in a first mammal and a second, non-syngeneic, second mammal, and of transplanting a graft derived from the first mammal in the second mammal while reducing both graft rejection and graft versus host disease, the method comprising:
administering antigens from said second mammal to said first mammal;
administering a non-myeloablative dose of at least one tyrphostin to said first mammal to selectively eliminate mammal lymphocytes responding to said antigens, thereby inducing immune tolerance in the first mammal to antigens of the second mammal;
administering antigens from said first mammal to said second mammal;
administering a non-myeloablative dose of at least one tyrphostin to said second mammal to selectively eliminate mammal lymphocytes responding to said antigens, thereby inducing immune tolerance in the second mammal to antigens of the first mammal; and
transplanting a graft derived from the first mammal in the second mammal.

86. The method of claim 85, wherein said graft is rich in immunocompetent lymphocytes.

87. The method of claim 86, wherein said graft is selected from the group consisting of bone marrow cells, small intestine and pancreatic islets.

88. The method of claim 85, further comprising, prior to, or concomitant with, administering said antigens from said second mammal:
administering at least one immunosuppressive agent to said first mammal in a non-myeloablative regimen sufficient to decrease the functional T lymphocyte population of said mammal.

89. The method of claim 86, wherein said antigens from said second mammal comprise one or more antigens selected from the group consisting of non-cellular antigens, cells, organs and tissues, either alive or killed.

90. The method of claim 89, wherein said antigens from said second mammal comprise hematopoietic cells.

91. The method of claim 88, wherein said at least one immunosuppressive agent is selected from the group consisting of an immunosuppressive drug, an alkylating agent, ionizing radiation and anti-leukocyte or anti-leukocyte function antibodies.

92. The method of claim 91, wherein said at least one immunosuppressive agent is a short-course total lymphoid irradiation (sTLI).

93. The method of claim 92, wherein said sTLI comprises administration of 1-12 doses of 200 cGy.

94. The method of claim 88, wherein said antigens from said second mammal comprise hematopoietic stem cells and said decrease of said T lymphocyte population is to a level permitting at least transient survival of said hematopoietic stem cells.

95. The method of claim 94, wherein said decrease of said T lymphocyte population is at least about 90%.

96. The method of claim 95, wherein said decrease of said T lymphocyte population is at least about 95%.

97. The method of claim 96, wherein said decrease of said T lymphocyte population is at least about 99%.

98. The method of claim 85, further comprising, prior to, or concomitant with, administering said antigens from said first mammal:
administering at least one immunosuppressive agent to said second mammal in a non-myeloablative regimen sufficient to decrease the functional T lymphocyte population of said mammal.

99. The method of claim 85, wherein said antigens from said first mammal comprise one or more antigens selected from the group consisting of non-cellular antigens, cells, organs and tissues, either alive or killed.

100. The method of claim 99, wherein said antigens from said first mammal comprise hematopoietic stem cells.

101. The method of claim 98, wherein said at least one immunosuppressive agent is selected from the group consisting of an immunosuppressive drug, an alkylating agent, ionizing radiation and anti-leukocyte or anti-leukocyte function antibodies.

102. The method of claim 101, wherein said at least one immunosuppressive agent is a short-course total lymphoid irradiation (sTLI).

103. The method of claim 102, wherein said sTLI comprises administration of 1-12 doses of 200 cGy.

104. The method of claim 98, wherein said antigens from said first mammal comprise hematopoietic stem cells and said decrease of said T lymphocyte population is to a level permitting at least transient survival of said hematopoietic stem cells.

105. The method of claim 104, wherein said decrease of said T lymphocyte population is at least about 90%.

106. The method of claim 105, wherein said decrease of said T lymphocyte population is at least about 95%.

107. The method of claim 106, wherein said decrease of said T lymphocyte population is at least about 99%.

108. The method of claim 85, further comprising, prior to said transplanting:
administering a preparation of hematopoietic stem cells from said first mammal to said non-syngeneic second mammal.

109. The method of claim 108, further comprising, following administering said preparation:
treating said first mammal with allogeneic cell therapy, said allogeneic cell therapy comprises infusing allogeneic lymphocytes from said donor into said host mammal.

110. The method of claim 108, wherein the blood of said second mammal comprises at least 20% cells of said first mammal.

111. The method of claim 85, wherein said second, non-syngeneic, mammal is an allogeneic mammal.

112. The method of claim 85, wherein said second, non-syngeneic, mammal is a xenogeneic mammal.

113. The method of claim 111, wherein said second mammal is a human patient.

114. The method of claim 113, wherein said human patient is a cancer patient.

115. The method of claim 112, wherein said second mammal is a human patient.

116. The method of claim 115, wherein said second mammal is a cancer patient.

117. The method of claim 85, wherein said at least one tyrphostin is of a family selected from the group consisting of quinoxalines, quinazolines, cyano-substituted acrylamides, cyano-substituted thioacrylamides, acrylonitriles, phenyl-substituted acrylonitriles, substituted anilines, benzoxazolones, tricyclic pyridones and tetracyclic pyridones.

118. The method of claim 85, wherein said at least one tyrphostin is selected from the group consisting of: N-benzyl-2-cyano-3-(3,4-dihydroxyphenyl)-acrylamide, N-benzyl-2-cyano-3-[3,4-dihydroxy-5-(3-phenylpropylsulfanylmethyl)-phenyl]-acrylamide, 4-amino-1-t-butyl-3-(p-toluyl)pyrazolo[3,4-d]pyrimidine, 4-amino-1-t-butyl-3-(p-chlorophenyl)pyrazolo[3,4-d]pyrimidine, 2-cyano-3-(3,4-dihydroxyphenyl)-N-(4-phenylbutyl)-acrylamide, 2-cyano-3-(3,4-dihydroxy-5-phenethylsulfanylmethyl-phenyl)-N-(4-phenylbutyl)-acrylamide, 2-(3-hydroxy-4-nitro-benzylidene)malononitrile, 4-(6,7-dimethoxyquinazoline-4-yl-amino)-benzamide, 4-(6,7-dimethoxyquinazoline-4-yl-amino)-phenol, 4-amino-1-t-butyl-3-(2-thiophene)pyrazolo[3,4-d]pyrimidine, 3-(3,5-dimethyl-H-pyrrol-2-yl-methylene)-2-oxo-2,3-dihydro-1H-indole-5-sulfonic acid dimethylamide, 4-amino-1-t-butyl-3-(3,4-dimethoxyphenyl)pyrazolo[3,4-d]pyrimidine, 2-cyano-3-(3,5-di-t-butyl-4-hydroxy phenyl)-thioacrylamide, 3-amino-5-[1-cyano-2-(1H-indol-3-yl)vinyl]-1H-pyrazole-4-carbonitrile, N-benzyl-2-cyano-3-(3,4-dihydroxy phenyl)-thioacrylamide, 2,3-dicyano-6-phenyl-pyridazine, 2-cyano-3-(3,4-dihydroxy-5-iodophenyl)-N-(4-phenyl propyl)-acrylamide, 2-(4-methoxy-benzylidene)malononitrile, and 2-cyano-3-(3,4-dihydroxyphenyl)-thioacrylamide.

119. A method of determining an activity of a tyrphostin in selective elimination of lymphocytes of a first mammal, the lymphocytes responding to antigens of a second, non-syngeneic, mammal, the method comprising:
stimulating hematopoietic cells of said first mammal with first antigens of said second mammal in a presence of and without said tyrphostin; and
thereafter exposing said hematopoietic cells of said first mammal to second antigens of said second mammal without said tyrphostin and measuring a response of said blood mononuclear cells of said first mammal to said antigens of said second mammal, thereby determining an activity of a tyrphostin in selective elimination of lymphocytes.

120. The method of claim 119, wherein said stimulating is in vitro, whereby said hematopoietic cells are isolated mononuclear cells or bone marrow cells.

121. The method of claim 119, wherein said stimulating is in vivo, whereby said hematopoietic cells are blood mononuclear cells and/or bone marrow cells.

122. The method of claim 121, wherein said exposing is in vivo or in vitro.

123. The method of claim 119, wherein said first antigens are isolated blood mononuclear cells or bone marrow cells.

124. The method of claim 119, wherein said second antigens are isolated blood mononuclear cells or bone marrow cells.

125. The method of claim 119, wherein said response is proliferation.

126. A method of determining an optimal concentration of a tyrphostin for selective elimination of lymphocytes of a first mammal, the lymphocytes responding to antigens of a second, non-syngeneic, mammal, the method comprising:
stimulating hematopoietic cells of said first mammal with first antigens of said second mammal in a presence of different concentrations of said tyrphostin; and thereafter
exposing said hematopoietic cells of said first mammal to second antigens of said second mammal without said tyrphostin and measuring a response of said blood mononuclear cells of said first mammal to said antigens of said second mammal, thereby determining an optimal concentration of a tyrphostin for selective elimination of lymphocytes.

127. The method of claim 126, wherein said stimulating is in vitro, whereby said hematopoietic cells are isolated mononuclear cells or bone marrow cells.

128. The method of claim 126, wherein said stimulating is in vivo, whereby said hematopoietic cells are blood mononuclear cells and/or bone marrow cells.

129. The method of claim 128, wherein said exposing is in vivo or in vitro.

130. The method of claim 126, wherein said first antigens are isolated blood mononuclear cells or bone marrow cells.

131. The method of claim 126, wherein said second antigens are isolated blood mononuclear cells or bone marrow cells.

132. The method of claim 126, wherein said response is proliferation.

133. A method of producing a non-human mammal/human hematopoietic mixed chimera, the method comprising:
administering antigens from a human to a non-human mammal;
administering a non-myeloablative dose of at least one tyrphostin to said non-human mammal to selectively eliminate lymphocytes responding to said antigens; and
administering a preparation of hematopoietic stem cells from said human to said mammal.

134. The method of claim 133, further comprising, prior to, or concomitant with, administering said antigens:
administering an immunosuppressive agent to said non-human mammal in a non-myeloablative regimen sufficient to decrease the functional T lymphocyte population of said mammal.

135. The method of claim 133, wherein said non-human mammal is a rodent.

136. The method of claim 133, wherein said non-human mammal is a pig.

137. A non-human mammal stably engrafted with human hematopoietic stem cells, the rodent constituting a hematopoietic mixed chimera.

138. A rodent stably engrafted with human hematopoietic stem cells, the rodent constituting a hematopoietic mixed chimera.

139. A pig stably engrafted with human hematopoietic stem cells, the pig constituting a hematopoietic mixed chimera.

140. A method of producing a first non-human mammal/second non-human mammal hematopoietic mixed chimera, the method comprising:
administering antigens from a first non-human mammal to a second non-human mammal;
administering a non-myeloablative dose of at least one tyrphostin to said second non-human mammal to selectively eliminate lymphocytes responding to said antigens; and
administering a preparation of hematopoietic stem cells from said first non-human mammal to said second non-human mammal.

141. A packaged pharmaceutical composition comprising, as an active ingredient, an effective amount of at least one tyrphostin and a pharmaceutically acceptable carrier, the pharmaceutical composition is packaged in a package and is identified in print associated with the package for use in an immune tolerance application.

142. The packaged pharmaceutical composition of claim 141, wherein said immune tolerance application comprises inducing immune tolerance in a first mammal to antigens of a second, non-syngeneic, mammal.

143. The packaged pharmaceutical composition of claim 142, wherein said immune tolerance application comprises:
administering antigens from said second mammal to said first mammal; and
administering a non-myeloablative dose of said at least one tyrphostin to said first mammal to selectively eliminate mammal lymphocytes responding to said antigens.

144. The packaged pharmaceutical composition of claim 141, wherein said immune tolerance application comprises inducing immune tolerance in a first mammal to antigens of a second, non-syngeneic, mammal, and transplanting a graft derived from the second mammal in the first mammal, while minimizing graft rejection.

145. The packaged pharmaceutical composition of claim 144, wherein said immune tolerance application comprises:
administering antigens from said second mammal to said first mammal;
administering a non-myeloablative dose of at least one tyrphostin to said first mammal to selectively eliminate mammal lymphocytes responding to said antigens; and
transplanting the graft in the first mammal.

146. The packaged pharmaceutical composition of claim 141, wherein said immune tolerance application comprises inducing immune tolerance in a first mammal to antigens of a second, non-syngeneic, mammal, and transplanting a graft derived from the first mammal in the second mammal while reducing graft-versus-host disease.

147. The packaged pharmaceutical composition of claim 146, wherein said immune tolerance application comprises:
administering antigens from said second mammal to said first mammal;
administering a non-myeloablative dose of at least one tyrphostin to said first mammal to selectively eliminate mammal lymphocytes responding to said antigens; and
transplanting the graft in the second mammal.

148. The packaged pharmaceutical composition of claim 141, wherein said immune tolerance application comprises inducing bilateral immune tolerance in a first mammal and a second, non-syngeneic, second mammal.

149. The packaged pharmaceutical composition of claim 148, wherein said immune tolerance application comprises:
administering antigens from said second mammal to said first mammal;
administering a non-myeloablative dose of at least one tyrphostin to said first mammal to selectively eliminate mammal lymphocytes responding to said antigens, thereby inducing immune tolerance in the first mammal to antigens of the second mammal;
administering antigens from said first mammal to said second mammal; and
administering a non-myeloablative dose of at least one tyrphostin to said second mammal to selectively eliminate mammal lymphocytes responding to said antigens, thereby inducing immune tolerance in the second mammal to antigens of the first mammal.

150. The packaged pharmaceutical composition of claim 141, wherein said immune tolerance application comprises inducing bilateral immune tolerance in a first mammal and a second, non-syngeneic, second mammal, and of transplanting a graft derived from the first mammal in the second mammal while reducing both graft rejection and graft versus host disease.

151. The packaged pharmaceutical composition of claim 150, wherein said immune tolerance application comprises:
administering antigens from said second mammal to said first mammal;
administering a non-myeloablative dose of at least one tyrphostin to said first mammal to selectively eliminate mammal lymphocytes responding to said antigens, thereby inducing immune tolerance in the first mammal to antigens of the second mammal;
administering antigens from said first mammal to said second mammal;
administering a non-myeloablative dose of at least one tyrphostin to said second mammal to selectively eliminate mammal lymphocytes responding to said antigens, thereby inducing immune tolerance in the second mammal to antigens of the first mammal; and
transplanting a graft derived from the first mammal in the second mammal.

152. The packaged pharmaceutical composition of claim 141, wherein said at least one tyrphostin is of a family selected from the group consisting of quinoxalines, quinazolines, cyano-substituted acrylamides, cyano-substituted thioacrylamides, acrylonitriles, phenyl-substituted acrylonitriles, substituted anilines, benzoxazolones, tricyclic pyridones and tetracyclic pyridones.

153. The packaged pharmaceutical composition of claim 141, wherein said at least one tyrphostin is selected from the group consisting of: N-benzyl-2-cyano-3-(3,4-dihydroxyphenyl)-acrylamide, N-benzyl-2-cyano-3-[3,4-dihydroxy-5-(3-phenylpropylsulfanylmethyl)-phenyl]-acrylamide, 4-amino-1-t-butyl-3-(p-toluyl)pyrazolo[3,4-d]pyrimidine, 4-amino-1-t-butyl-3-(p-chlorophenyl)pyrazolo[3,4-d]pyrimidine, 2-cyano-3-(3,4-dihydroxyphenyl)-N-(4-phenylbutyl)-acrylamide, 2-cyano-3-(3,4-dihydroxy-5-phenethylsulfanylmethyl-phenyl)-N-(4-phenylbutyl)-acrylamide, 2-(3-hydroxy-4-nitro-benzylidene)malononitrile, 4-(6,7-dimethoxyquinazoline-4-yl-amino)-benzamide, 4-(6,7-dimethoxyquinazoline-4-yl-amino)-phenol, 4-amino-1-t-butyl-3-(2-thiophene)pyrazolo[3,4-d]pyrimidine, 3-(3,5-dimethyl-H-pyrrol-2-yl-methylene)-2-oxo-2,3-dihydro-1H-indole-5-sulfonic acid dimethylamide, 4-amino-1-t-butyl-3-(3,4-dimethoxyphenyl)pyrazolo[3,4-d]pyrimidine, 2-cyano-3-(3,5-di-t-butyl-4-hydroxy phenyl)-thioacrylamide, 3-amino-5-[1-cyano-2-(1H-indol-3-yl)vinyl]-1H-pyrazole-4-carbonitrile, N-benzyl-2-cyano-3-(3,4-dihydroxy phenyl)-thioacrylamide, 2,3-dicyano-6-phenyl-pyridazine, 2-cyano-3-(3,4-dihydroxy-5-iodophenyl)-N-(4-phenyl propyl)-acrylamide, 2-(4-methoxy-benzylidene)malononitrile, and 2-cyano-3-(3,4-dihydroxyphenyl)-thioacrylamide.

154. A method of inducing an immune tolerance to a specific antigen in a mammal, the method comprising:
administering said specific antigen to the mammal; and
administering a non-myeloablative dose of at least one tyrphostin to said mammal, to selectively eliminate mammal lymphocytes responding to said specific antigen.

155. The method of claim 154, wherein said specific antigen is a self antigen.

156. The method of claim 154, wherein said specific antigen comprise factor 8 proteins.

157. The method of claim 154, wherein said specific antigen is associated with an autoimmune disease or a disease having an autoimmune component.

158. The method of claim 157, wherein said autoimmune disease is selected from the group consisting of multiple sclerosis, lupus erythematosus and rheumatoid arthritis.

159. The method of claim 154, wherein said at least one tyrphostin is of a family selected from the group consisting of quinoxalines, quinazolines, cyano-substituted acrylamides, cyano-substituted thioacrylamides, acrylonitriles, phenyl-substituted acrylonitriles, substituted anilines, benzoxazolones, tricyclic pyridones and tetracyclic pyridones.

160. The method of claim 154, wherein said at least one tyrphostin is selected from the group consisting of: N-benzyl-2-cyano-3-(3,4-dihydroxyphenyl)-acrylamide, N-benzyl-2-cyano-3-[3,4-dihydroxy-5-(3-phenylpropylsulfanyhnethyl)-phenyl]-acrylamide, 4-amino-1-t-butyl-3-(p-toluyl)pyrazolo[3,4-d]pyrimidine, 4-amino-1-t-butyl-3-(p-chlorophenyl)pyrazolo[3,4-d]pyrimidine, 2-cyano-3-(3,4-dihydroxyphenyl)-N-(4-phenylbutyl)-acrylamide, 2-cyano-3-(3,4-dihydroxy-5-phenethylsulfanylmethyl-phenyl)-N-(4-phenylbutyl)-acrylamide, 2-(3-hydroxy-4-nitro-benzylidene)malononitrile, 4-(6,7-dimethoxyquinazoline-4-yl-amino)-benzamide, 4-(6,7-dimethoxyquinazoline-4-yl-amino)-phenol, 4-amino-1-t-butyl-3-(2-thiophene)pyrazolo[3,4-d]pyrimidine, 3-(3,5-dimethyl-H-pyrrol-2-yl-methylene)-2-oxo-2,3-dihydro-1H-indole-5-sulfonic acid dimethylamide, 4-amino-1-t-butyl-3-(3,4-dimethoxyphenyl)pyrazolo[3,4-d]pyrimidine, 2-cyano-3-(3,5-di-t-butyl-4-hydroxy phenyl)-thioacrylamide, 3-amino-5-[1-cyano-2-(1H-indol-3-yl)vinyl]-1H-pyrazole-4-carbonitrile, N-benzyl-2-cyano-3-(3,4-dihydroxy phenyl)-thioacrylamide, 2,3-dicyano-6-phenyl-pyridazine, 2-cyano-3-(3,4-dihydroxy-5-iodophenyl)-N-(4-phenyl propyl)-acrylamide, 2-(4-methoxy-benzylidene)malononitrile, and 2-cyano-3-(3,4-dihydroxyphenyl)-thioacrylamide.
